SEQUENCE LISTING (33 sequences)

SEQ ID NO: 1 - protein, 183 aa, Hepatitis B Virus Subtype AYW
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 2 - protein, 185 aa, Hepatitis B Virus Subtype ADW
MDIDPYKEFGATVELLSFLPSDFFPSVRDLLDTASALYREALESPEHCSPHHTALRQAILCWGELMTLATWVGNNLQDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 3 - protein, 185 aa, Hepatitis B Subtype ADW2
MDIDPYKEFGATVELLSFLPSDFFPSVRDLLDTASALYREALESPEHCSPHHTALRQAILCWGELMTLATWVGNNLEDPASRDLVVNYVNTNVGLKIRQLLWFHISCLTFGRETVLEYLVSFGVWIRTPPAYRPPNAPILSTLPETTVVRRRDRGRSPRRRTPSPRRRPSQSPRRRRSQSRESQC

SEQ ID NO: 4 - protein, 183 aa, Hepatitis B Subtype ADYW
MDIDPYKEFGATVELLSFLPSDFFPSVRDLLDTAAALYRDALESPEHCSPHHTALRQAILCWGDLMTLATWVGTNLEDPASRDLVVSYVNTNVGLKFRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 5 - protein, 183 aa, Woodchuck Hepatitis Virus
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQC

SEQ ID NO: 6 - protein, 217 aa, Ground Squirrel Hepatitis Virus
MYLFHLCLVFACVPCPTVQASKLCLGWLWDMDIDPYKEFGSSYQLLNFLPLDFFPDLNALVDTAAALYEEELTGREHCSPHHTAIRQALVCWEELTRLITWMSENTTEEVRRIIVDHVNNTWGLKVRQTLWFHLSCLTFGQHTVQEFLVSFGVWIRTPAPYRPPNAPILSTLPEHTVIRRRGGSRAARSPRRRTPSPRRRRSQSPRRRRSQSPASNC

SEQ ID NO: 7 - DNA, 549 bp, Hepatitis B Virus Subtype AYW
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgt

SEQ ID NO: 8 - DNA, 555 bp, Hepatitis B Virus B Subtype ADW
atggacattgacccttataaagaatttggagctactgtggagttactctcgtttttgccttctgacttctttccttccgtacgagatctcctagacaccgcctcagctctgtatcgagaagccttagagtctcctgagcattgctcacctcaccatactgcactcaggcaagccattctctgctggggggaattgatgactctagctacctgggtgggtaataatttgcaagatccagcatccagagatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgt

SEQ ID NO: 9 - DNA, 554 bp, Hepatitis B Subtype ADW2
atggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctcgctggggggaattgatgactctagctacctgggtgggtaataatttggaagatccagcatctagggatcttgtagtaaattatgttaatactaacgtgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctccatcgccgcgtcgcagaagatctcaatctcgggaatctcaatgt

SEQ ID NO: 10 - DNA, 549 bp, Hepatitis B Subtype ADYW
atggacattgacccttataaagaatttggagctactgtggagttactctcgtttttgccttctgacttctttccttccgtacgagatcttctagataccgccgcagctctgtatcgggatgccttagagtctcctgagcattgttcacctcaccatactgcactcaggcaagcaattctttgctggggagacttaatgactctagctacctgggtgggtactaatttagaagatccagcatctagggacctagtagtcagttatgtcaacactaatgtgggcctaaagttcagacaattattgtggtttcacatttcttgtctcacttttggaagagaaacggttctagagtatttggtgtcttttggagtgtggattcgcactcctccagcttatagaccaccaaatgcccctatcctatcaacgcttccggagactactgttgttagacgacgaggcaggtcccctagaagaagaactccctcgcctcgcagacgaagatctcaatcgccgcgtcgcagaagatctcaatctcgggaatctcaatgt

SEQ ID NO: 11 - DNA, 549 bp, Woodchuck Hepatitis Virus
atggctttggggcatggacatagatccttataaagaatttggttcatcttatcagttgttgaattttcttcctttggacttctttcctgatcttaatgctttggtggacactgctactgccttgtatgaagaagaactaacaggtagggaacattgctctccgcaccatacagctattagacaagctttagtatgctgggatgaattaactaaattgatagcttggatgagctctaacataacttctgaacaagtaagaacaatcattgtaaatcatgtcaatgatacctggggacttaaggtgagacaaagtttatggtttcatttgtcatgtctcactttcggacaacatacagttcaagaatttttagtaagttttggagtatggatcaggactccagctccatatagacctcctaatgcacccattctctcgactcttccggaacatacagtcattaggagaagaggaggtgcaagagcttctaggtcccccagaagacgcactccctctcctcgcaggagaagatctcaatcaccgcgtcgcag

SEQ ID NO: 12 - DNA, 651 bp, Ground Squirrel Hepatitis Virus
atgtatctttttcacctgtgccttgtttttgcctgtgttccatgtcctactgttcaagcctccaagctgtgccttggatggctttgggacatggacatagatccctataaagaatttggttcttcttatcagttgttgaattttcttcctttggacttttttcctgatctcaatgcattggtggacactgctgctgctctttatgaagaagaattaacaggtagggagcattgttctcctcatcatactgctattagacaggccttagtgtgttgggaagaattaactagattaattacatggatgagtgaaaatacaacagaagaagttagaagaattattgttgatcatgtcaataatacttggggacttaaagtaagacagactttatggtttcatttatcatgtcttacttttggacaacacacagttcaagaatttttggttagttttggagtatggattagaactccagctccttatagaccacctaatgcacccattttatcaactcttccggaacatacagtcattaggagaagaggaggttcaagagctgctaggtccccccgaagacgcactccctctcctcgcaggagaaggtctcaatcaccgcgtcgcagacgctctcaatctccagcttccaactgc

SEQ ID NO: 13 - protein, 20 aa, Artificial Sequence
  Synthetic Construct
MNNATFNYTNVNPISHIRGS

SEQ ID NO: 14 - DNA, 32 bp, Artificial Sequence
  Synthetic Construct
ctgttgacaattaatcatcggctcgtataatg

SEQ ID NO: 15 - DNA, 87 bp, Artificial Sequence
  Synthetic Construct
ggaattccggcgaaactgctgaaagaacgtggcttttttggcgcgattgcgggctttctggagctcggcagcggtgatgaaggggga

SEQ ID NO: 16 - DNA, 29 bp, Artificial Sequence
  Synthetic Construct
ttgacaattaatcatcggctcgtataatg

SEQ ID NO: 17 - DNA, 66 bp, Influenza B Virus
atgaacaacgcgacctttaactataccaacgtgaacccgattagccatattcgtggatccgaactc

SEQ ID NO: 18 - protein, 19 aa, Influenza B Virus
PAKLLKERGFFGAIAGFLE

SEQ ID NO: 19 - protein, 23 aa, Influenza B Virus
PAKLLKERGFFGAIAGFLEGSGC

SEQ ID NO: 20 - protein, 19 aa, Influenza B Virus
NNATFNYTNVNPISHIRGS

SEQ ID NO: 21 - protein, 12 aa, Influenza B Virus
LEPFQILSISGC

SEQ ID NO: 22 - protein, 24 aa, Influenza B Virus
FWRGENGRKTRSAYERMCNILKGK

SEQ ID NO: 23 - protein, 22 aa, Influenza B Virus
LRVLSFIRGTKVSPRGKLSTRG

SEQ ID NO: 24 - protein, 22 aa, Influenza B Virus
SLVGIDPFKLLQNSQVYSLIRP

SEQ ID NO: 25 - protein, 24 aa, Influenza B Virus
AVKGVGTMVMELIRMIKRGINDRN

SEQ ID NO: 26 - protein, 179 aa, Artificial Sequence
  Synthetic Construct
MDIDPYKEFGATVELLSFLPSDFFPSVRDLLDTASALYREALESPEHCSPHHTALRQAILCWGELMTLATWVGVNLEDGIPAKLLKERGFFGAIAGFLELGSGDEGGPASRDLVVSYVNTNMGLKFRQLLWFHISCLTFGRETVIEYLVSFGVWIRTPPAYRPPNAPILSTLPETTVVC

SEQ ID NO: 27 - protein, 150 aa, Hepatitis B Virus
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVC

SEQ ID NO: 28 - protein, 183 aa, Artificial Sequence
  Derived from Influenza B NBe sequence
MNNATFNYTNVNPISHIRGSELDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPC

SEQ ID NO: 29 - protein, 169 aa, Artificial Sequence
  Derived from Influenza B NBe sequence
MNNATFNYTNVNPISHIRGSELDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVC

SEQ ID NO: 30 - DNA, 5090 bp, Artificial Sequence
  Synthetic Construct
gcggataacaatttcacataaggaggaaaaaaccatgaacaacgcgacctttaactataccaacgtgaacccgattagccatattcgtggcagcggcggaattcaaaaagagctcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgtttgctagtaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtga

SEQ ID NO: 31 - DNA, 5105 bp, Artificial Sequence
  Synthetic Construct
tagaggatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacataaggaggaaaaaaccatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatggaattccggcgaaactgctgaaagaacgtggcttttttggcgcgattgcgggctttctggagctcggcagcggtgatgaagggggaccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgtttgctagtaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcg

SEQ ID NO: 32 - DNA, 4239 bp, Artificial Sequence
  Synthetic Construct
gatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacataaggaggaaaaaaccatgaacaacgcgacctttaactataccaacgtgaacccgattagccatattcgtggatccgaactcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttcgtcgacgaggccgctcccctcgtcggcgcactccctcgccttgttagtaagcttatcgatgataagctgtcaaacatgagaattaacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagggtcgacaattcgcgcgcgaaggcgaagcggcatgcatttacgttgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgtcgaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaagcatgcgaaacgaccgtcatcctgtctcttgatca

SEQ ID NO: 33 - protein, 29 aa, Artificial Sequence
  Synthetic construct
GIPAKLLKERGFFGAIAGFLELGSGDEGG

(19) United States
(12) Patent Application Publication
(10) Pub. No.: US 2011/0110973 A1
(43) Pub. Date: May 12, 2011

(54) Influenza B Vaccines

(75) Inventors: Kirill Kalnin, Pelham, NH (US); Ashley Birkett, Bethesda, MD (US); Patricia Londono-Arcila, Boston, MA (US); Svetlana Stegalkina, Pelham, NH (US); Yanhua Yan, Westford, MA (US)

(73) Assignee: Sanofi Pasteur Biologics Co., Cambridge, MA (US)

(21) Appl. No.: 12/866,064

(22) PCT Filed: Feb. 9, 2009
(86) PCT No.: PCT/US09/00838

§ 371 Date: Jan. 14, 2011

Related U.S. Application Data
(60) Provisional application No. 61/065,327, filed on Feb. 9, 2008.

(51) Int. Cl. A61K 39/295; C07K 14/11; C07H 21/00; A61P 31/16; A61P 37/04

(52) U.S. Cl. 424/202.1; 530/350; 536/23.72

(57) ABSTRACT

This invention relates to immunogenic compositions including influenza B virus sequences, and methods of using such compositions.

### FIELD OF THE INVENTION

This invention relates to immunogenic compositions including influenza B virus sequences, and methods of using such compositions.

### BACKGROUND OF THE INVENTION

An influenza pandemic occurs when a new influenza virus subtype appears, against which the global population has little or no immunity. During the 20^{th} century, influenza pandemics caused millions of deaths, social disruption, and profound economic losses worldwide. Influenza experts agree that another pandemic is likely to happen, but it is unknown when. The level of global preparedness at the moment when a pandemic strikes will determine the public health and economic impact of the disease. As of today, the WHO estimates that there will be at least several hundred million outpatient visits, more than 25 million hospital admissions, and several million deaths globally, within a very short period. Infection by influenza virus is responsible for 20,000 to 40,000 deaths and over 100,000 hospitalizations each year in the United States alone (Simonsen et al., J. Infect. Dis. 181:831-837, 2000).

There are two influenza viruses of public health concern, A and B. Influenza A virus replicates in a wide range of avian and mammalian hosts. Subtypes are defined based on the immunological specificity of the hemagglutinin (HA) and neuraminidase (NA) envelope proteins. Two genetically and antigenically distinct lineages of influenza B virus are cocirculating in humans, as represented by the B/Yamagata/16/88 and B/Victoria/2/87 viruses (Ferguson et al., Nature 422:428-443, 2003). Although the spectrum of disease caused by influenza B virus is generally milder than that caused by influenza A virus, severe illness requiring hospitalization is still frequently observed with influenza B infection (Murphy et al. (Ed.), Fields Virology, 3^{rd} ed., Lippincott-Raven, Philadelphia, Pa.).

Current approaches to influenza vaccination, relying on the induction of influenza virus-neutralizing antibody responses to the HA protein, will be inefficient in the face of a pandemic, because of the long time required for identification of the virus, and construction and manufacture of a suitable vaccine. Thus, alternatives to traditional HA-based vaccines must be investigated.

### SUMMARY OF THE INVENTION

The invention provides polypeptides including hepatitis B core protein sequences and influenza B virus sequences (e.g., hemagglutinin precursor protein sequences, NB sequences, NBe sequences, M2 sequences, or M2e sequences). The hepatitis B core protein sequences may optionally include a carboxy-terminal truncation (e.g., a carboxy-terminal truncation after amino acid 149, 150, 163, or 164). The influenza B virus sequences can be inserted in the major immunodominant region (MIR) of the hepatitis B core protein sequences (e.g., in the region of amino acids 75-83 of the hepatitis B core protein sequences) or can be inserted at the amino terminus of the hepatitis B core protein. Alternatively, the hepatitis B core and influenza B virus sequences can be chemically-linked. Further, the recombinant hepatitis B virus core (HBc) protein can include Domains I, II, III, and IV as described herein.

The invention also includes virus-like particles that include any of the polypeptides described herein, optionally in combination with hepatitis B core sequences lacking the insertion or chemical linkage of influenza B virus sequences. Further, the invention includes nucleic acid molecules encoding the polypeptides described herein, as well as pharmaceutical compositions that include one or more of the polypeptides or the virus-like particles described herein, optionally, in combination with an adjuvant. The pharmaceutical compositions can include pharmaceutically acceptable carriers or diluents (e.g., water or saline) or may be in lyophilized form.

Also included in the invention are methods of inducing an immune response to influenza virus B in a subject, by administering to the subject any of the polypeptides, virus-like particles, nucleic acid molecules, and/or pharmaceutical compositions described herein. Such methods can be carried out with subjects that do not have, but are at risk of acquiring, an influenza B virus infection, or subjects that already have such an infection. Further, the methods can include the administration of one or more different immunological agents against an influenza virus or other pathogens.

The invention provides several advantages. For example, as discussed above, current approaches to influenza vaccines, relying on the induction of influenza virus-neutralizing antibody responses to the HA protein, will be inefficient in the face of newly emerging strains, because of the long time required for virus identification, construction and manufacture of a suitable vaccine. The present invention provides alternatives to traditional HA-based vaccines, which are recombinant vaccines based on conservative epitopes (e.g., BHA0 and NBe), and are intended to provide protection against all B strains of the influenza. These vaccines will not need to be renewed annually, and thus can be stockpiled for use in the event of an influenza pandemic. The fusions of the invention are also readily made by recombinant means, leading to increased safety and efficiency, relative to inactivated vaccines.

Other features and advantages of the invention will be apparent from the following detailed description, the drawings, and the claims.

### BRIEF DESCRIPTION OF THE DRAWINGS

FIG. 1, shown in two panels as FIG. 1A and FIG. 1B, provides an alignment of six published sequences for mammalian HBc proteins from six viruses (SEQ ID NO:1-6). The human viral sequences are of the ayw subtype (Galibert et al., Nature 281:646-650, 1983), the adw subtype (Ono et al., Nucleic Acids Res. 11(6):1747-1757, 1983), the adw2 subtype (Valenzuela et al., Animal Virus Genetics, Field et al. eds., Academic Press, New York (1980) pages 57-70), and the adyw subtype (Pasek et al., Nature 282:575-579, 1979). Also shown is the sequence of the woodchuck virus (Galibert et al., J. Virol. 41:51-65, 1982) and that of the ground squirrel (Seeger et al., J. Virol. 51:367-375, 1984).

FIG. 2 illustrates an example of a single plasmid expression system for expression of hybrid particles (3010). Both wild type HBc and HBC-HA0 monomers are produced from the same plasmid, from different tac promoters. An HA0 peptide was inserted into the major immunodominant region (MIR) of HBc, between amino acids 78 and 79.

FIG. 3 shows that the HA0 peptide is immunogenic in various forms. Immune responses generated by two immunizations of the indicated VLPs (see Table 1) are shown.

FIG. 4 shows that the HA0 loop insertion is superior to the chemically linked peptide at inducing antibodies reactive with infected cells. MDCK cell staining is shown with pooled (8 mice per group) sera samples derived from mice immunized with either 3004 or 1843-HA0. Left panel—cells infected with influenza B (Memphis) virus; Right panel—uninfected cells. Green staining (relatively light staining in black and white) signifies recognition of virus on the surface of the cells by the given serum samples.

FIG. 5 shows that the BHA0 loop insertion induces antibodies that are cross-reactive with recombinant and native HA. Antibodies generated against 3004 and 1843-BHA0 are immunoreactive with recombinant and native HA. Left panel—reactivity of antibodies (ELISA results) with recombinant HA; Right panel—reactivity with Fluvirin® (native HA). Gp9—mice immunized with Fluvirin® vaccine; Gp8 and Gp5—mice immunized with 3004 or 1843-BHA, respectively.

FIG. 6 shows the results of a challenge model experiment with mouse-adapted influenza B/Memphis/12/97 (M15). This strain was passaged 6 times in mice (LD50>10⁵ PFU), mice were challenged via the intranasal route with 10⁵ PFU, and body weight was followed daily for 14 days (n=6). Separate groups of challenged mice were sacrificed on days 3 and 4 for determination of virus load in lungs.

FIG. 7 shows morbidity data from challenge experiment 1. Mice were immunized subcutaneously on three occasions (days 0, 21, and 42) with the indicated particles (10 μg each) and QS21 adjuvant (10 μg) (total volume per mouse 100 μl) and challenged via the intranasal route with 1.5×10⁵ of adapted influenza B/Memphis/12/97 (M15) strain on day 63. Fluvirin® was given intramuscularly, in two sites, 50 μl per site, to a total 3 μg HA of influenza B. Weight measurements were followed daily.

FIG. 8 shows lung load data from challenge experiment 1. Mice were immunized subcutaneously on three occasions (days 0, 21, and 42) with the indicated particles (10 μg each) and QS21 adjuvant (10 μg) (total volume per mouse 100 μl) and challenged via the intranasal route with 1.5×10⁵ of adapted influenza B/Memphis/12/97 (M15) strain on day 63. Lung counts were followed daily.

FIG. 9 shows morbidity data from challenge experiment 2. Mice were immunized subcutaneously on two occasions (days 0 and 21) with the indicated particles (10 μg each) and QS21 adjuvant (10 μg) (total volume per mouse 100 μl) and challenged via the intranasal route with 1.5×10³ of adapted influenza B/Memphis/12/97 (M15) strain on day 42. The weights of the mice were monitored daily.

FIG. 10 shows lung load data from challenge experiment 2 (day 4). Mice were immunized subcutaneously on two occasions (days 0 and 21) with the indicated particles (10 μg each) with QS21 adjuvant (10 μg) (total volume per mouse 100μl) and challenged via the intranasal route with 1.5×10³ of adapted influenza B/Memphis/12/97 (M15) strain on day 42.

FIG. 11 shows the principle scheme of HBc-NBe 3026 (302) and 3002 (300) constructs. In addition, chemical conjugates of HBc1843 particles were prepared as described in U.S. Pat. No. 6,231,864 B1. The latter particles were designated as 1843-NBe.

FIG. 12 shows that the genetic fusion (3002) is superior to the chemical conjugate (1843-NBe) for NBe immunogenicity.

FIG. 13 shows that two genetic HBc-Nbe constructs, 3002 and 3026, resulted in similar immunogenicity. Constructs 3002 (150 mer) and 3026 (165 mer) induced similar antibody titers, but responses against 3002 are more consistent (shorter HBc).

FIG. 14 shows that immune responses in mice to NBe genetically inserted into HBc (3002) are dose-dependent. 2imm=2 immunizations; 3imm=3 immunizations.

FIG. 15 shows the results of immunogenicity studies of a BM2e-KLH conjugate and an HBc-BM2e chemical conjugate.

FIG. 16 shows schematic illustrations of plasmid maps and corresponding sequences of construct 3002 as described herein. The BHA0 and flanking sequence is shown to encode MNNATFNYTNVNPISHIRGS. HBc sequences are shown between the EcoRI and HindIII restriction sites.

FIG. 17 shows schematic illustrations of plasmid maps and corresponding sequences of construct 3004 as described herein. The ptac promoter is shown to include the sequence CTGTTGACAATTAATCATCGGCTCGTATAATG. The HBc sequences are shown to be between the NcoI and EcoRI sites, and also downstream from the SacI site and through the HindIII site. Inserted HA0 and flanking sequences are shown by

GGAATTCCGGCGAAACTGCTGAAAGAACGTGGCTTTTTTGGCGCGATTG
CGGGCTTTCTGGAGCTCGGCAGCGGTGATGAAGGGGGA.

FIG. 18 shows schematic illustrations of plasmid maps and corresponding sequences of construct 3026 as described herein. The ptac promoter is shown to include the sequence TTGACAATTAATCATCGGCTCGTATAATG. The HBc sequences are shown to be between BamHI and HindIII sites. Inserted NBe sequences are shown to include

ATGAACAACGCGACCTTTAACTATACCAACGTGAACCCGATTAGCCATA
TTCGTGGATCCGAACTC.

FIGS. 19-24 show schematic illustrations of certain constructs of the invention, as well as purification schemes and results.

### DETAILED DESCRIPTION

The present invention relates to biological fusions of immunogenic influenza B virus peptides to hepatitis B core (HBc) protein sequences, and virus-like particles (VLPs) formed from these fusions. Influenza B sequences that can be used in the invention include HA0, NB, and M2 sequences, as well as fragments (e.g., NBe and M2e) and variants thereof as described herein. In addition, the invention relates to chemical conjugates of influenza B sequences, such as HA0, NB, and M2, as well as fragments (e.g., NBe and M2e) and variants thereof, to HBc. Further, the invention includes pharmaceutical compositions (e.g., inocula and vaccines) including the fusions and conjugates described herein, the use of such compositions in immunization methods, nucleic acid molecules encoding the fusions, vectors containing the nucleic acid molecules, and cells containing the vectors. The fusions, conjugates, compositions, and methods of the invention are first generally described below, and then additional details are provided, followed by experimental examples. In addition, reference is made to U.S. Pat. No. 7,361,352, which is incorporated herein by reference, for additional details concerning the construction and use of HBc fusions and VLPs that can be applied to practice of the present invention.

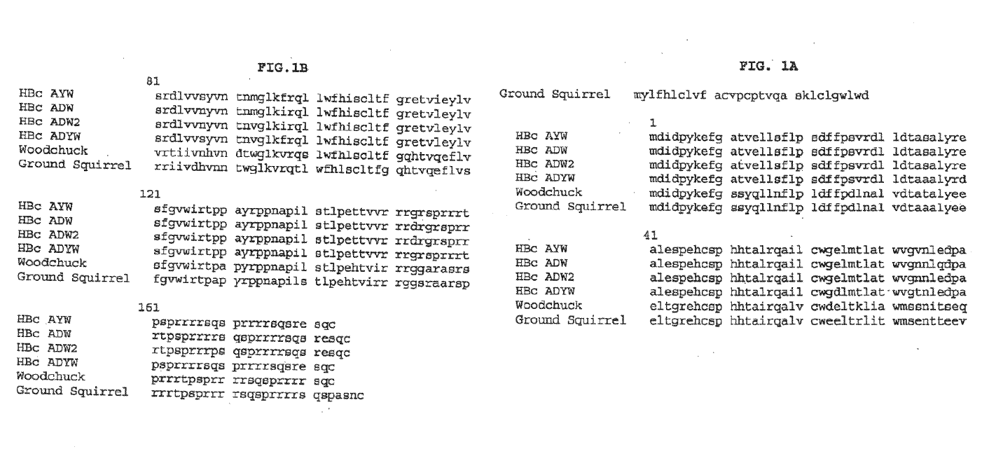

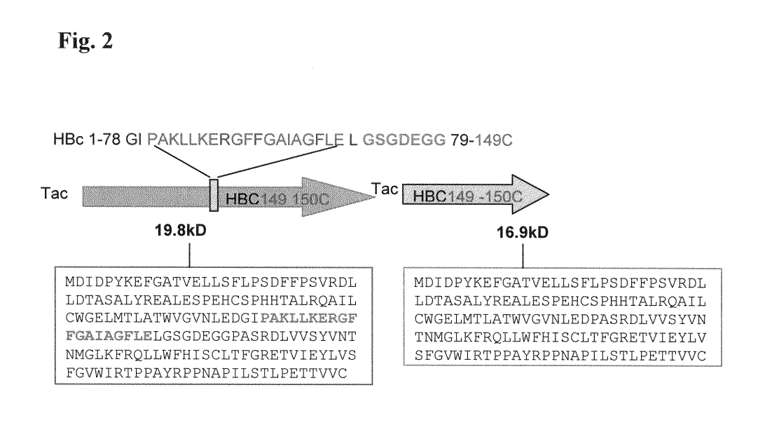

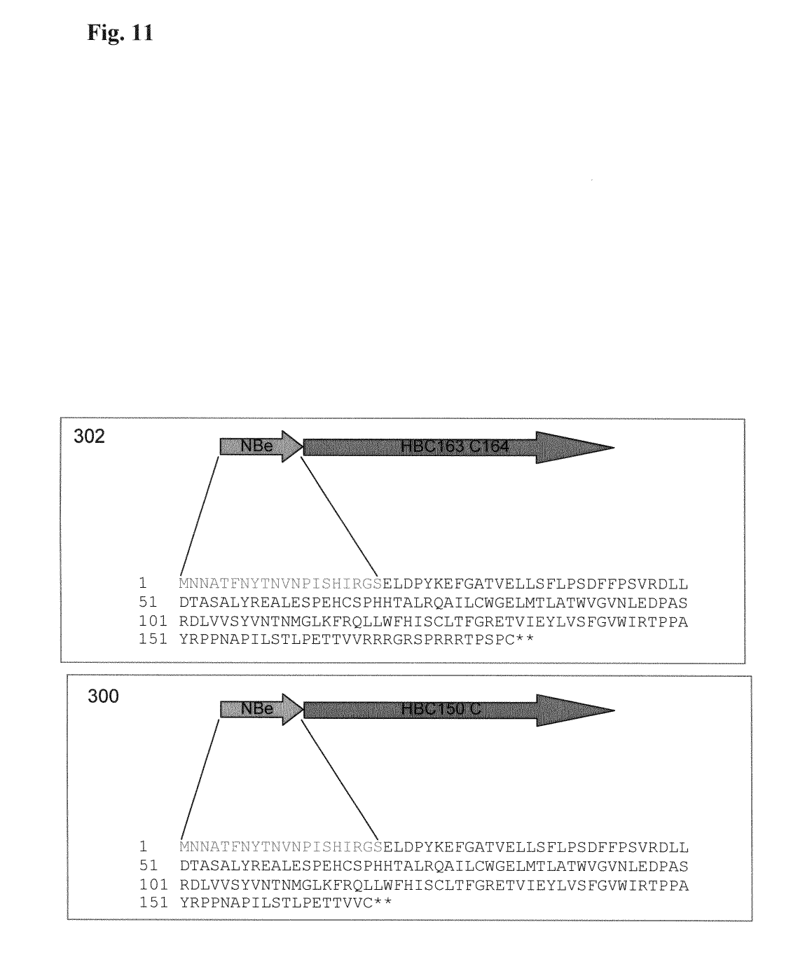

Hepatitis B core sequences that can be used in the invention include full-length sequences of, e.g., mammalian (e.g., HBc ayw, HBc adw, HBc adw2, and HBc adwy), woodchuck, and ground squirrel, and other HBc (see, e.g., FIG. 1), as well as truncated sequences (e.g., carboxy-terminal truncated sequences, which are truncated at, e.g., amino acid 149, 150, 163, or 164; see, e.g., FIG. 11; and/or amino-terminal truncated sequences, which lack 1, 2, 3, or 4 amino acids from the amino terminal end of HBc if no additional sequences are added to the amino terminal region (e.g., NB, M2, or HA0 sequences) or more deleted sequences, e.g., 1, 2, 3, 4, 5, or 6 amino acids if such sequences are added; and/or internal deletions), and variants thereof.

Influenza B virus sequences (or fragments or variants thereof) can be inserted within the HBc sequences and/or at either end of the HBc sequences as described herein. For example, sequences can be inserted into the major immunodominant region (MIR) of HBc, which is in the area of about amino acid positions 70-90, and more specifically 75-83, of HBc. The insertions into the MIR region can thus be between any amino acids in this region (e.g., 70-71, 71-72, 73-74, 75-76, 76-77, 77-78, 78-79, 79-80, 80-81, 81-82, 82-83, 83-84, 84-85, 85-86, 87-88, 88-89, or 89-90), or can be present in the place of deletions of, e.g., 1-20, 2-18, 3-15, 4-12, 5-10, or 6-8 amino acids in this region. A specific example, which is described further below, includes an insertion of influenza B virus sequences between amino acids 78 and 82 of HBc. Use of this type of internal insertion may be particularly beneficial in the case of inserted HA0 sequences, as such an internal insertion may provide a favorable conformation with respect to presentation of sequences that are normally internal to a protein, such as HA0 sequences, to the immune system. However, this type of insertion can be used for other antigens (e.g., NB (e.g., NBe) and M2 (e.g., M2e) sequences). In another example, insertions are made at the amino-terminus of the HBc protein. This configuration may be favorable with respect to presentation of sequences that are normally terminal sequences, such as NB (e.g., NBe) and M2 (e.g., M2e) sequences. However, this type of insertion can also be used for other antigens, such as HA0 sequences, as described herein. Additional details of this type of construction of the invention are provided below.

The inserted HA0, NB, and M2 sequences can comprise single fragments or epitopes or can be in the form of polytopes, in which multiple (e.g., 2, 3, 4, 5, or more) repeats of the fragment or epitope are inserted. The sequences of the inserted material can be from any strain of influenza B virus to which the induction of an immune response is desired, or a consensus sequence derived from comparisons of sequences of different strains. Examples of sequences that can be used in the invention include PAKLLKERGFFGAIAGFLE (HA0), PAKLLKERGFFGAIAGFLEGSGC (HA0), NNATFNYTNVNPISHIRGS (NBe), and LEPFQILSISGC (M2e), as well as truncations or expansions of these sequences by, e.g., 1, 2, 3, 4, 5, 6, 7, 8, 9, or 10 amino acids within the sequences or on either or both ends. Further, if desired, linker sequences can be used between inserted sequences and each other (in the case of polytopes) and/or between inserted sequences and HBc sequences. For example, glycine-rich sequences can be used (see below for a specific, non-limiting example). The invention also includes use of variants of the above-noted sequences, which include, e.g., one or more (e.g., 2, 3, 4, 5, 6, 7, 8, 9, 10, or more) conservative amino acid substitutions, deletions, or insertions. “Conservative amino acid substitution” as used herein denotes that an amino acid residue has been replaced by another, biologically similar residue. Examples of conservative substitutions include the substitution of one hydrophobic residue such as isoleucine, valine, leucine, or methionine for another, or the substitution of one polar residue for another such as between arginine and lysine, between glutamic acid and aspartic acid, or between glutamine and asparagine and the like.

The polypeptides described herein can be used in the form of virus-like particles, as described herein, which may Optionally include, in addition to HBc sequences including insertions and/or chemically linked influenza B sequences, HBc sequences lacking insertions (see below). The use of HBc sequences including insertions (or chemical fusions) and HBc sequences lacking insertions (or chemical fusions) together to make HBc VLPs results in the production of so-called “hybrid VLPs.” As discussed further below, hybrid VLPs can be made by expression of both types of HBc molecules (with and without insert or added influenza sequences) from the same plasmid, generally resulting in about a 1:1 ratio of the two types of HBc molecules being present in the VLPs. In other approaches, the two types of HBc molecules can be expressed from different plasmids. In these approaches, the different types of HBc molecules can be present at about a 1:1 ratio or at different ratios, as determined to be appropriate by those of skill in the art. For example, the different types of molecules can be present at ratios ranging from 1:10 to 10:1. Further, in addition to including the two different types of HBc noted above, hybrid VLPs can also include multiple (e.g., 2, 3, 4, 5, or more) different influenza antigens, as described herein. Thus, for example, a VLP of the invention can include mixtures of HBc fusion proteins, with some including one or more of HA0, NB (e.g., NBe), and/or M2 (e.g., M2e) sequences, inserted at any one or more of the loci described herein, optionally with HBc sequences lacking insertions/fused sequences, and further optionally with HBc sequences including other inserted/fused sequences (e.g., influenza A sequences, e.g., M2 (e.g., M2e sequences), influenza A HA0 sequences, influenza A neuraminidase sequences, etc.).

As described further below, the HBc-based compositions of the invention can be administered as single component agents, or in combination with other active ingredients. Such additional active ingredients may be, for example, influenza A immunogenic compositions such as vaccines (e.g., any of the vaccines described in U.S. Pat. No. 7,361,352, the contents of which are incorporated by reference) and/or additional immunogenic agents directed against influenza B virus. The immunogenic agents can include influenza A and/or B hemagglutinin, neuraminidase, M2 (e.g., M2e), and/or NB (e.g., NBe)-derived antigens, or fragments thereof

The compositions of the invention can optionally include one or more adjuvants, as described further below, such as an aluminum compound (alum, aluminum hydroxide), muramyl dipeptide analogs, QS21, and other adjuvants known to those of skill in the art.

Also as discussed further below, the compositions of the invention can be administered to subjects by, e.g., parenteral (e.g., intramuscular, subcutaneous, or intradermal) routes. In addition, administration may be carried out by of intranasal or oral formulations. The subjects treated according to the invention include human patients (e.g., adults, children, infants, and elderly patients), as well as animals (e.g., livestock, domestic pets, and birds), for which sequences may have to be adapted, depending upon influenza B strains infecting the animals.

Additional details concerning HBc-based fusions of the invention and their uses are provided below, followed by experimental examples. Numerals utilized herein in conjunction with descriptions of HBc chimeras indicate the position in the HBc ayw amino acid residue sequence (FIG. 1) at which one or more residues has been added to or deleted from the sequence, regardless of whether additions or deletions to the amino acid residue sequence are present. Thus, HBc149 indicates that the chimera ends at residue 149, whereas HBc149+C150 indicates that the same chimera contains a cysteine residue at HBc position 150 relative to the sequence of HBc ayw. HBc149 remains indicated as such, even if sequences are added to or removed from, e.g., amino terminal and/or internal regions of the sequence.

As discussed above, the invention includes an immunogen and a vaccine or pharmaceutical composition comprising that immunogen against the influenza B virus. An immunogen of the invention can be a particle comprised of recombinant hepatitis B virus core (HBc) protein chimeric molecules with a length of about 150 to about 375 amino acids, e.g., about 150 to 235 amino acids, that contain four peptide-linked amino acid sequence domains from the N-terminus that are denoted herein in various examples as Domains I, II, III, and IV. Optionally, the molecules can include one to three cysteine residues at or near the N-terminus and/or the C-terminus of the chimera and/or one or more (e.g., two to four) polypeptides containing 6 to about 24 (e.g., about 8-23, 10-19, or 12-15) residues of the influenza B HA0, NB (e.g., NBe), and/or M2 (e.g., M2e) polypeptides, as defined herein, present either peptide-bonded or chemically fused to the chimeric molecule.

In one example, an immunogenic chimeric particle includes Domains I, II, III, and IV described as follows.

(a) Domain I comprises about 75 to about 160 amino acid residues having a sequence that includes at least the sequence of the residues of position 4 through about position 75 of HBc. One to three cysteine residues are optionally also present at a position in the chimeric molecule, at or near the amino terminus, or at a position of about one to about −55, e.g., to about −30, or to about −20, relative to the N-terminus of HBc. Such N-terminal cysteine residues can be present within a sequence other than that of the pre-core sequence of HBc.

(b) Domain II comprises about zero to about 60 amino acid residues peptide-bonded to about residue 75. This sequence includes (i) zero to all 10 of the residues of a sequence of HBc from HBc position 76 through 85 (or zero to all of the residues of a sequences of HBc from HBc positions 70-90) peptide-bonded to (ii) an optional sequence of about 6 to about 48 or more residues that constitute one or more repeats of 6 to about 24 (e.g., about 8-23, 10-19, or 12-15) residues of an influenza B polypeptide (e.g., an HA0, NB (e.g., NBe), or M2 (e.g., M2e) sequences).

(c) Domain III is an HBc sequence from about position 86 through about position 135 that is peptide-bonded to about residue 85 of Domain II.

(d) Domain IV comprises (i) the residues of positions 136 through 140 plus up to sixteen residues of an HBc amino acid residue sequence from position 141 through 156 peptide-bonded to the residue of position 135 of Domain III, (ii) zero to three cysteine residues, (iii) optionally fewer than four arginine or lysine residues, or mixtures thereof adjacent to each other, and (iv) up to about 100 amino acid residues in a sequence heterologous to HBc from position 156 to the C-terminus. Thus, Domain IV contains at least the 5 residues of positions 136-140.

A chimeric molecule of the invention can, in various examples, (i) contain up to about 10 percent conservatively substituted amino acid residues in the HBc sequence, and (ii) self-assemble into particles that are substantially free of binding to nucleic acids upon expression in a host cell. Further, a particle of the invention can optionally include HBc molecules with N-terminal cysteines, and those particles may be more stable on formation than are particles formed from an otherwise identical HBc chimera that lacks the N-terminal cysteine residue(s) or in which an N-terminal cysteine residue present in the chimera molecule is replaced by another residue. One example of a chimeric molecule of the invention contains a cysteine residue that is present at a position of about −50 to about +1 relative to the N-terminus of HBc as is illustrated in FIG. 1. The concept of a negative amino acid position is usually associated with a leader sequence such as the pre-core sequence of HBc. That concept is used similarly here in that one can simply align a given chimeric molecule sequence with that of a sequence of FIG. 1 to determine the position of the chimera that corresponds to that of the starting methionine residue of position +1 of HBc. Inasmuch as amino acid residue sequences are normally shown from left to right and in the direction from N-terminus to C-terminus, any aligned chimeric molecule residue to the left of the position occupied by the HBc start methionine has a negative position. A cysteine residue can occur at any position about fifty or twenty residues to the left of the aligned start methionine of HBc up to the position corresponding to that start methionine.

In examining the length of an HBc chimera of the invention, such a recombinant protein can have a length of about 150 to about 325 amino acid residues, e.g., about 150 to about 235 amino acid residues, or about 170 to about 215 amino acid residues. These differences in length arise primarily from changes in the length of Domains I, II, and IV, and particularly the number of insert polypeptides present and whether a C-terminal sequence heterologous to HBc is present.

An N-terminal sequence peptide-bonded to one of the first five N-terminal residues of HBc can contain a sequence of up to about 40 residues that are heterologous to HBc; i.e., a portion of a pre-core sequence can be present in a o contemplated chimeric molecule. Exemplary sequences include influenza A or B, B cell or T cell epitopes such as are discussed hereinafter.

Domain I can include the sequence of residues of positions 1-, 2-, 3-, or 4-through position 75 of HBc. Domain I also optionally contains one to three added cysteine residue(s) and also can optionally include two to four sequences of about 6 to about 24 (e.g., about 8-23, 10-19, or 12-15) residues of the sequence of an inserted

Influenza B sequence as described herein peptide-bonded at the amino-terminus as discussed herein below. Domain I therefore can contain a deletion of at least the methionine residue of position 1 of HBc and can include deletions of the residues at HBc positions 2, 3, and 4.

The optional one to three cysteine residues noted above can be present at a position in the chimeric molecule of about one to about -55, -30, or -20, relative to the N-terminus of HBc. Thus, using the sequence of HBc ayw (FIG. 1) as a reference point, the N-terminal cysteine residue(s) can be located in the chimeric molecule at a position that corresponds to the methionine at position 1 of the sequence, or at a position up to about 50 residues upstream from that position. In various examples, an N-terminal cysteine is located at a position of about one to about minus 14 relative to position 1 of the HBc ayw sequence.

The one or more N-terminal cysteine residues can be present within a sequence other than that of the pre-core sequence of HBc. As was noted previously, the HBeAg molecule contains the pre-core sequence that includes a cysteine residue. That molecule does not form particles, whereas particles are generally desired herein. Thus, although an N-terminal cysteine residue can be adjacent to a pre-core sequence, such a residue is not typically present within a pre-core sequence or a contemplated chimeric molecule.

Domain I can have a length of about 160 residues, e.g., a length of about 95 to about 145 amino acid residues, and can include at least one, e.g., one to four, or two to three influenza B polypeptide sequences, as described herein.

Domain II, which is peptide-bonded to about residue 75 of Domain I, contains about zero to about 60 amino acid residues. This Domain includes zero (none), at least 4, or at least 8 residues, through all 10 of the HBc sequence residues of about positions 76 through about position 85. Domain II also optionally includes a sequence of about 6 to about 48 (e.g., about 8-23, 10-19, or 12-15) residues that constitute one or more repeats of an influenza virus B sequence as described herein. The influenza B polypeptide sequence, when present, can be peptide-bonded between HBc residues 78 and 82, in one example.

Domain III contains the sequence of HBc from about position 86 through about position 135 peptide-bonded at its N-terminus to about residue 85.

The fourth domain, Domain IV, comprises (i) the residues of positions 136 through 140 plus up to sixteen residues of an HBc amino acid residue sequence from position 141 through position 156, e.g., nine residues through 149 peptide-bonded to the residue of about position 135 of Domain III, (ii) optionally zero to three cysteine residues, such as one cysteine residue, (iii) fewer than four arginine or lysine residues, or mixtures thereof adjacent to each other, and (iv) up to about 100, 50, or 25 amino acid residues, in a sequence heterologous to HBc from position 164 or from position 156 to the C-terminus.

In one example, Domain IV contains up to fourteen residues of an HBc sequence from position 136 through position 149 peptide-bonded to residue 135; i.e., an HBc sequence that begins with the residue of position 136 that can continue through position 149. Thus, if the residue of position 148 is present, so is the sequence of residues of positions 136 through 147, or if residue 141 is present, so is the sequence of residues of positions 136 through 140.

Domain IV can also contain zero to three cysteine residues and those Cys residues are present within about 30 residues of the carboxy-terminus (C-terminus) of the chimeric molecule. In one example, one cysteine (Cys) residue is present, and that Cys can be present as the carboxy-terminal (C-terminal) residue, unless an influenza T cell epitope is present as part of Domain IV (see below). When such a T cell epitope is present, the Cys can be within the C-terminal last five residues of the HBc chimera.

The presence of the above-described N-terminal cysteine residue(s) can provide an enhancement of the ability of the chimeric molecules to form stable immunogenic particles (discussed hereinafter). Thus, HBc chimeric immunogens in general tend to form particles that stay together upon collection and initial purification as measured by analytical size exclusion chromatography or SDS-PAGE analysis, as described in U.S. Pat. No. 7,361,352.

Particles that additionally contain one or more C-terminal cysteine residues exhibit enhanced stability in formation and also toward decomposition on aging, with some particles containing both N- and C-terminal cysteines usually exhibiting greater stability in either measure than those particles having only an added cysteine at either the N- or C-terminus. A particle containing a N-terminal cysteine residue is also typically prepared in greater yield than is a particle assembled from a chimeric molecule lacking a N-terminal cysteine.

Domain IV can contain fewer than four arginine or lysine residues, or mixtures thereof adjacent to each other. Arginine and lysines are present in the C-terminal region of HBc that extends from position 156 through the C-terminus of the native molecule. That region is sometimes referred to as the protamine or arginine-rich region of the molecule and binds nucleic acids. HBc chimeric molecules and particles of the invention are typically substantially free of bound nucleic acids, as can be readily determined by a comparison of the absorbance of the particles in aqueous solution measured at both 280 and 260 nm; i.e., a 280/260 absorbance ratio (see, e.g., U.S. Pat. No. 7,361,352).

Although the T cell help afforded by HBc is highly effective in enhancing antibody responses (i.e., B cell-mediated response) to carried immunogenic sequences following vaccination, HBc does not activate influenza-specific T cells, except in restricted individuals for whom a B cell epitope containing sequence also contains a T cell epitope. To help ensure universal priming of influenza-specific T helper cells, in addition to B cells, one or more influenza-specific T helper epitopes can be incorporated into a contemplated immunogen and is located in Domain IV of the immunogen.

A plurality of T cell epitopes can be present in Domain IV or another B cell epitope can be present. In an exemplary practice, Domain IV has up to about 50 residues in a sequence heterologous to HBc. In one example, that sequence is up to about 25 residues and includes a T cell epitope.

Th epitopes derived from the influenza nucleoprotein (NP 206-229), which is broadly reactive in humans (HLA-DR1, HLA-DR2, HLA-DRw13) (Brett et al., J. Immunol. 147(3):984-991, 1991) and also functional in BALB/c mice are contemplated for use as T cell epitopes herein. Additional influenza Th epitopes can also be used, such as NP 341-362, NP 297-318, and NP 182-205 (Brett et al., J. Immunol. 147(3):984-991, 1991); these sequences can be, e.g., linked in series at the C-terminus of the influenza B peptide-expressing particle. These illustrative sequences are provided below.

| NP Position | Sequence |
|---|---|
| 206-229 | FWRGENGRKTRSAYERMCNILKGK |
| 341-362 | LRVLSFIRGTKVSPRGKLSTRG |
| 297-318 | SLVGIDPFKLLQNSQVYSLIRP |
| 182-205 | AVKGVGTMVMELIRMIKRGINDRN |

HBc chimeric molecules of the invention are typically present in and are used in an immunogen or vaccine as a self-assembled particle. These particles are comprised of 180 to 240 chimera molecules that separate into protein molecules in the presence of disulfide reducing agents such as 2-mercaptoethanol and denaturing reagents such as SDS. The individual molecules are bound together into the particle by protein-protein interactions, and these interactions are stabilized by the presence of disulfide bonds. These particles are similar to the particles observed in patients infected with HBV, but are non-infectious. Upon expression in various prokaryotic and eukaryotic hosts, the individual recombinant HBc chimeric molecules assemble in the host into particles that can be readily harvested from the host cells.

In addition to the above-described N- and C-truncations and insertion of influenza sequences, chimeric molecules of the invention can also contain conservative substitutions in the amino acid residues that constitute HBc Domains I, II, III, and IV. Conservative substitutions are as defined before. More rarely, a “nonconservative” change, e.g., replacement of a glycine with a tryptophan is contemplated. Analogous minor variations can also include amino acid deletions or insertions, or both. Guidance in determining which amino acid residues can be substituted, inserted, or deleted without abolishing biological activity can be found using computer programs well known in the art, for example LASERGENE software (DNASTAR Inc., Madison, Wisc.).

The HBc portion of a chimeric molecule of the present invention (the portion having the HBc sequence that has other than a sequence of an added epitope, or heterologous residue(s) that are a restriction enzyme artifact) can have the amino acid residue sequence at positions 2 through 149 of subtype ayw that is shown in FIG. 1, when present. Other examples are the corresponding amino acid residue sequences of subtypes adw, adw2, and adyw, which are also shown in FIG. 1, and the sequences of woodchuck and ground squirrel at aligned positions 2 through 149, which are the last two sequences of FIG. 1. Corresponding nucleic acid molecule sequences are provided below. Further, portions of different sequences from different mammalian HBc proteins can be used together in a single chimera.

When the HBc portion of a chimeric molecule of the invention has other than a sequence of a mammalian HBc molecule at positions 2 through 156 or through position 149, when present, because one or more conservative substitutions has been made, typically no more than 10 percent, 5 percent, or 3 percent of the amino acid residues are substituted as compared to the HBc ayw sequence of FIG. 1 from position 2 through 149 or 156. A contemplated chimera of 149 HBc residues can therefore contain up to about 15 or 16 (or 7 or 8, or up to 5) residues that are different from those of the Hbc ayw sequence of FIG. 1 at positions 2 through 149. Where an HBc sequence is truncated further at one or both termini, the number of substituted residues is proportionally different. Deletions elsewhere in the molecule are considered conservative substitutions for purposes of calculation so that if, for example, Domain I were to have a C-terminus at position 133 instead of 135, two residues (134 and 135) would be presumed to be present for purposes of calculation.

### Chimera Preparation

Chimeric immunogens of the invention are prepared using the well-known techniques of recombinant DNA technology. Thus, nucleic acid sequences that encode particular polypeptide sequences are added and deleted from the precursor sequence that encodes HBV.

As was noted above, the HBc immunodominant loop is usually recited as being located at about positions 70 to 90, in particular, positions 75 through 85, from the amino-terminus (N-terminus) of the intact protein. The influenza B sequence can be placed into that immunodominant loop sequence of Domain II. That placement can substantially eliminate the HBc immunogenicity and antigenicity of the HBc loop sequence, while presenting the influenza B sequence in an extremely immunogenic position in the assembled chimeric particles.

One of two well-known strategies is particularly useful for placing the influenza B sequence into the loop sequence at a desired location, such as between residues 78 and 79. A first strategy is referred to as replacement, in which DNA that codes for a portion of the loop is excised and replaced with DNA that encodes an influenza B sequence. The second strategy is referred to as insertion, in which an influenza B sequence is inserted between adjacent residues in the loop.

Site-directed mutagenesis using the polymerase chain reaction (PCR) can be used in one exemplary replacement approach to provide a chimeric HBc DNA sequence that encodes a pair of different restriction sites, e.g., EcoRI and SacI, one near each end of the immunodominant loop-encoding DNA. Exemplary residues replaced are 76 through 81 (also see above). The loop-encoding section is excised, an influenza B sequence flanked on each side by appropriate HBc sequence residues is ligated into the restriction sites, and the resulting DNA is used to express the HBc chimera.

Alternatively, a single restriction site or two sites can be encoded into the region, the DNA cut with a restriction enzyme(s) to provide sticky or blunt ends, and an appropriate sticky- or blunt-ended heterologous DNA segment ligated into the cut region. Examples of this type of sequence replacement into HBc can be found in Schodel et al., (1991) F. Brown et al. eds., Vaccines 91, Cold Spring Harbor Laboratory, Cold Spring Harbor, N.Y., pp. 319-325; Schodel et al., Behring Inst. Mitt. (98):114-119, 1997; and Schodel et al., J. Exp. Med., 180(3):1037-1044, 1994.

In an illustrative example of the insertion strategy, site-directed mutagenesis is used to create two restriction sites adjacent to each other and between codons encoding adjacent amino acid residues, such as those at residue positions 78 and 79. This technique adds twelve base pairs that encode four amino acid residues (two for each restriction site) between formerly adjacent residues in the HBc loop. Upon cleavage with the restriction enzymes, ligation of the DNA coding for the illustrative influenza B sequence and expression of the DNA to form HBc chimers, the HBc loop amino acid sequence is seen to be interrupted on its N-terminal side by the two residues encoded by the 5′ restriction site, followed toward the C-terminus by the influenza B sequence, followed by two more heterologous, non-loop residues encoded by the 3′ restriction site and then the rest of the loop sequence. This same strategy can also be used for insertion into Domain IV of a T cell epitope or one or more cysteine residues that are not a part of a T cell epitope.

For example, a DNA sequence that encodes a C-terminal truncated HBc sequence (HBc149) is engineered to contain adjacent EcoRI and Sad sites between residues 78 and 79. Cleavage of that DNA with both enzymes provides one fragment that encodes HBc positions 1-78 3′-terminated with an EcoRI sticky end, whereas the other fragment has a 5′-terminal Sad sticky end and encodes residues of positions 79-149. Ligation of a synthetic nucleic acid having a 5′ AATT overhang followed by a sequence that encodes a desired influenza B sequence and a AGCT 3′overhang provides a HBc chimeric sequence that encodes an influenza B sequence flanked on each side by two heterologous residues (GI and EL, respectively) between residues 78 and 79, while destroying the EcoRI site and preserving the Sad site.

A similar strategy can be used for insertion of a C-terminal cysteine-containing sequence. In this example, EcoRI and HindIII restriction sites are engineered into the HBc DNA sequence after amino acid residue position 149. After digestion with

EcoRI and HindIII, a synthetic DNA having the above-noted AATT 5′overhang followed by a T cell epitope-encoding sequence, a stop codon, and a 3′ AGCT overhang are ligated into the digested sequence to form a sequence that encodes HBc residues 1-149 followed by two heterologous residues (GI), the stop codon and the HindIII site.

PCR amplification using a forward primer having a Sad restriction site followed by a sequence encoding HBc beginning at residue position 79, followed by digestion with Sad and HindIII provide a sequence encoding HBc positions 79-149 plus the two added residues and the T cell epitope at the C-terminus. Digestion of that construct with Sad and ligation provides the complete gene encoding a recombinant HBc chimeric immunogen having the sequence, from the N-terminus, of HBc positions 1-78, two added residues, the influenza B sequence, two added residues,

HBc positions 79-149, two added residues, and the T cell epitope.

It is noted that the use of two heterologous residues on either side of (flanking) a heterologous immunogenic sequence containing B cell or T cell epitopes is a matter of convenience. As a consequence, one can also use zero to three or more added residues that are not part of the HBc sequence on either or both sides of an inserted sequence. One or both ends of the insert and HBc nucleic acid can be “chewed back” with an appropriate nuclease (e.g., S1 nuclease) to provide blunt ends that can be ligated together. Added heterologous residues that are neither part of the inserted B cell or T cell epitopes nor a part of the HBc sequence are not counted in the number of residues present in a recited Domain.

It is also noted that one can also synthesize all or a part of a desired recombinant HBc chimeric nucleic acid using well-known synthetic methods as is discussed and illustrated in U.S. Pat. No. 5,656,472 for the synthesis of the 177 base pair DNA that encodes the 59 residue ribulose bis-phosphate carboxylase-oxygenase signal peptide of Nicotiana tabacum. For example, one can synthesize

Domains I and II with a blunt or sticky end that can be ligated to Domains III and IV to provide a construct that expresses a contemplated HBc chimera that contains zero added residues to the N-terminal side of the influenza B sequence and zero to three added residues on the C-terminal side or at the Domain II/III junction or at some other desired location.

A nucleic acid sequence (segment) that encodes a previously described HBc chimeric molecule or a complement of that coding sequence is also contemplated herein. Such a nucleic acid segment is present in isolated and purified form in some embodiments. In living organisms, the amino acid residue sequence of a protein or polypeptide is directly related via the genetic code to the deoxyribonucleic acid (DNA) sequence of the gene that codes for the protein. Thus, through the well-known degeneracy of the genetic code additional DNAs and corresponding RNA sequences (nucleic acids) can be prepared as desired that encode the same chimeric amino acid residue sequences, but are sufficiently different from a before-discussed gene sequence that the two sequences do not hybridize at high stringency, but do hybridize at moderate stringency.

High stringency conditions can be defined as comprising hybridization at a temperature of about 50°-−55° C. in 6× SSC and a final wash at a temperature of 68° C. in 1-3× SSC. Moderate stringency conditions comprise hybridization at a temperature of about 50° C. to about 65° C. in 0.2 to 0.3 M NaCl, followed by washing at about 50° C. to about 55° C. in 0.2× SSC, 0.1% SDS (sodium dodecyl sulfate).

A nucleic sequence (DNA sequence or an RNA sequence) that (1) itself encodes, or its complement encodes, a chimeric molecule including an HBc portion from residue position 1 through 136 that, when present, is that of SEQ ID NOs: 1, 2, 3, 4, 5, or 6 (FIGS. 1) and (2) can hybridize with a DNA sequence of SEQ ID NOs:7, 8, 9, 10, 11, or 12 at least at moderate stringency (discussed above); and (3) having an HBc sequence that shares at least 80 percent, at least 90 percent, at least 95 percent, or 100 percent identity with a DNA sequence of SEQ ID NOs: 7, 8, 9, 10, 11, or 12, is defined as a DNA variant sequence. As is well-known, a nucleic acid sequence is expressed when operatively linked to an appropriate promoter in an appropriate expression system as discussed elsewhere herein.

An analog or analogous nucleic acid (DNA or RNA) sequence that encodes a contemplated chimeric molecule is also within this invention. A chimeric analog nucleic acid sequence or its complementary nucleic acid sequence encodes a HBc amino acid residue sequence that is at least 80 percent, at least 90 percent, or at least 95 percent identical to the HBc sequence portion from residue position 1 through residue position 136 shown in SEQ ID NOs: 1, 2, 3, 4, 5, and 6. This DNA or RNA is referred to herein as an “analog of” or “analogous to” a sequence of a nucleic acid of SEQ ID NOs:7, 8, 9, 10, 11, or 12, and hybridizes with the nucleic acid sequence of SEQ ID NOs: 7, 8, 9, 10, 11, or 12, or their complements herein under moderate stringency hybridization conditions. A nucleic acid that encodes an analogous sequence, upon suitable transfection and expression, also produces a contemplated chimera.

Different hosts often have preferences for a particular codon to be used for encoding a particular amino acid residue. Such codon preferences are well known and a DNA sequence encoding a desired chimeric sequence can be altered, using in vitro mutagenesis for example, so that host-preferred codons are utilized for a particular host in which the protein is to be expressed. In addition, one can also use the degeneracy of the genetic code to encode the HBc portion of a sequence of SEQ ID NOs:1, 2, 3, 4, 5 or 6 that avoids substantial identity with a DNA of SEQ ID NOs: 7, 8, 9, 10, 11, or 12 or their complements. Thus, a useful analogous DNA sequence need not hybridize with the nucleotide sequences of SEQ ID NOs:7, 8, 9, 10, 11, or 12, or a complement under conditions of moderate stringency, but can still provide a chimeric molecule of the invention.

A recombinant nucleic acid molecule such as a DNA molecule, comprising a vector operatively linked to an exogenous nucleic acid segment (e.g., a DNA segment or sequence) that defines a gene that encodes a chimera of the invention, as discussed above, and a promoter suitable for driving the expression of the gene in a compatible host organism, is also contemplated in this invention. More particularly, also contemplated is a recombinant DNA molecule that comprises a vector comprising a promoter for driving the expression of the chimera in host organism cells operatively linked to a DNA segment that defines a gene for the HBc portion of a chimera or a DNA variant that has at least 90 percent identity to the HBc gene of SEQ ID NOs:7, 8, 9, 10, 11, or 12, and hybridizes with that gene under moderate stringency conditions.

Further included in the invention is a recombinant DNA molecule that comprises a vector containing a promoter for driving the expression of a chimera in host organism cells operatively linked to a DNA segment that is an analog nucleic acid sequence that encodes an amino acid residue sequence of a HBc chimera portion that is at least 80 percent identical, at least 90 percent identical, or at least 95 percent identical to the HBc portion of a sequence of SEQ ID NOs:1, 2, 3, 4, 5, or 6. That recombinant DNA molecule, upon suitable transfection and expression in a host cell, provides a chimeric molecule of the invention.

It is noted that because of the 30 amino acid residue N-terminal sequence of ground squirrel HBc does not align with any of the other HBc sequences, that sequence and its encoding nucleic acid sequences and their complements are not included in the above percentages of identity, nor are the portions of nucleic acid that encode that 30-residue sequence or its complement used in hybridization determinations. Similarly, sequences that are truncated at either or both of the HBc N- and C-termini are not included in identity calculations, nor are those sequences in which residues of the immunodominant loop are removed for insertion of a heterologous epitope. Thus, only those HBc-encoding bases or HBc sequence residues that are present in a chimeric molecule are included and compared to an aligned nucleic acid or amino acid residue sequence in the identity percentage calculations.

Inasmuch as the coding sequences for the gene disclosed herein is illustrated in SEQ ID NOs:7, 8, 9, 10, 11, or 12, isolated nucleic acid segments, such as DNA sequences, variants and analogs thereof can be prepared by in vitro mutagenesis, as is well known in the art and discussed in Current Protocols In Molecular Biology, Ausabel et al. eds., John Wiley & Sons (New York: 1987) p. 8.1.1-8.1.6, that begin at the initial ATG codon for a gene and end at or just downstream of the stop codon for each gene. Thus, a desired restriction site can be engineered at or upstream of the initiation codon, and at or downstream of the stop codon so that other genes can be prepared, excised and isolated.

As is well known in the art, as long as the required nucleic acid, illustratively DNA, sequence is present (including start and stop signals), additional base pairs can usually be present at either end of the segment and that segment can still be utilized to express the protein. This, of course, presumes the absence in the segment of an operatively linked DNA sequence that represses expression, expresses a further product that consumes the protein desired to be expressed, expresses a product that consumes a wanted reaction product produced by that desired enzyme, or otherwise interferes with expression of the gene of the DNA segment.

Thus, as long as the DNA segment is free of such interfering DNA sequences, a DNA segment of the invention can be about 500 to about 15,000 base pairs in length. The maximum size of a recombinant DNA molecule, particularly an expression vector, is governed mostly by convenience and the vector size that can be accommodated by a host cell, once all of the minimal DNA sequences required for replication and expression, when desired, are present. Minimal vector sizes are well known.

DNA segments that encode the above-described chimeras can be synthesized by chemical techniques, for example, the phosphotriester method of Matteucci et al., J. Am. Chem. Soc. 103:3185, 1981. By chemically synthesizing the coding sequence, any desired modifications can be made simply by substituting the appropriate bases for those encoding the native amino acid residue sequence.

A HBc chimera of the invention can be produced (expressed) in a number of transformed host systems, typically host cells although expression in acellular, in vitro systems is also included in the invention contemplated. These host cellular systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus or tobacco mosaic virus) or with bacterial expression vectors (e.g., Ti plasmid); and appropriately transformed animal cell systems such as CHO or COS cells. The invention is not limited by the host cell employed.

DNA segments containing a gene encoding the HBc chimera can be obtained from recombinant DNA molecules (plasmid vectors) containing that gene. Vectors capable of directing the expression of a chimeric gene into the protein of a HBc chimeric is referred to herein as an “expression vector.” An expression vector typically contains expression control elements including a promoter. The chimera-coding gene is operatively linked to the expression vector to permit the promoter sequence to direct RNA polymerase binding and expression of the chimera-encoding gene. Useful in expressing the polypeptide coding gene are promoters that are, e.g., inducible, viral, synthetic, and constitutive as described by Poszkowski et al., EMBO J. 3:2719, 1989, and Odell et al., Nature 313:810, 1985, as well as temporally regulated, spatially regulated, and spatiotemporally regulated promoters as described in Chua et al., Science 244:174-181, 1989.

One example of a promoter for use in prokaryotic cells such as E. coli is the Rec 7 promoter that is inducible by exogenously supplied nalidixic acid. Another example of a promoter is present in plasmid vector JHEX25 (available from Promega) that is inducible by exogenously supplied isopropyl-β-D-thiogalacto-pyranoside (IPTG). Another promoter, the tac promoter, which is used in the experimental examples described herein (see below), is present in plasmid vector pKK223-3 and is also inducible by exogenously supplied IPTG. The pKK223-3 plasmid can be expressed in a number of E. coli strains, such as XL-1, TB1, BL21, and BLR, using about 25 to about 100 μM IPTG for induction.

Expression of chimeric molecules in other microbes such as Salmonella, such as S. typhi and S. typhimurium and S. typhimurium-E. coli hybrids, yeasts such as S. cerivisiae and Pichia pastoris, in mammalian cells such as Chinese hamster ovary (CHO) cells, in both monocot and dicot plant cells generally and particularly in dicot plant storage organs such as a root, seed or fruit as where an oral vaccine or inoculum is desired, and in insect cells such as those of S. frugiperda cells or Trichoplusia by use of Autographa californica nuclear polyhedrosis virus (AcNPV) or baculovirus as known in the art (see, e.g., WO 02/14478 A2).

A variety of methods have been developed to operatively link DNA to vectors via complementary cohesive termini or blunt ends. For instance, complementary homopolymer tracts can be added to the DNA segment to be inserted into the vector DNA. The vector and DNA segment are then joined by hydrogen bonding between the complementary homopolymeric tails to form recombinant DNA molecules.

Alternatively, synthetic linkers containing one or more restriction endonuclease sites can be used to join the DNA segment to the expression vector, as noted above. The synthetic linkers are attached to blunt-ended DNA segments by incubating the blunt-ended DNA segments with a large excess of synthetic linker molecules in the presence of an enzyme that is able to catalyze the ligation of blunt-ended DNA molecules, such as bacteriophage T4 DNA ligase.

Thus, the products of the reaction are DNA segments carrying synthetic linker sequences at their ends. These DNA segments are then cleaved with the appropriate restriction endonuclease and ligated into an expression vector that has been cleaved with an enzyme that produces termini compatible with those of the synthetic linker. Synthetic linkers containing a variety of restriction endonuclease sites are commercially available from a number of sources including New England BioLabs, Beverly, Mass. A desired DNA segment can also be obtained using PCR technology in which the forward and reverse primers contain desired restriction sites that can be cut after amplification so that the gene can be inserted into the vector. Alternatively PCR products can be directly cloned into vectors containing T-overhangs (Promega Corp., A3600, Madison, Wisc.) as is well known in the art.

The expressed chimeric protein self-assembles into particles within the host cells, whether in single cells or in cells within a multicelled host. The particle-containing cells are harvested using standard procedures, and the cells are lysed using a French pressure cell, lysozyme, sonicator, bead beater or a microfluidizer (Microfluidics International Corp., Newton Mass.). After clarification of the lysate, particles are precipitated with 45% ammonium sulfate, resuspended in 20 mM sodium phosphate, pH 6.8 and dialyzed against the same buffer. The dialyzed material is clarified by brief centrifugation and the supernatant subjected to gel filtration chromatography using Sepharose® CL-4B. Particle-containing fractions are identified, subjected to hydroxyapatite chromatography, and reprecipitated with ammonium sulfate prior to resuspension, dialysis, sterile filtration, and storage at −70° C.

### Inocula and Vaccines

The above-described recombinant HBc chimeras, typically in particulate form, are dissolved or dispersed in an immunogenic effective amount in a pharmaceutically acceptable vehicle composition (e.g., an aqueous liquid or solution) to form an inoculum or a vaccine. When administered to a host animal in need of immunization or in which antibodies are desired to be induced such as a mammal (e.g., a mouse, dog, goat, sheep, horse, bovine, monkey, ape, or human) or bird (e.g., a chicken, turkey, duck or goose), an inoculum induces antibodies that immunoreact with the influenza B sequence present in the immunogen. In a vaccine, those induced antibodies also believed to immunoreact in vivo with (bind to) the virus or virally-infected cells and protect the host from a pathogenic influenza infection. A composition that is a vaccine in one animal can be an inoculum for another host, as where the antibodies are induced in a second host that is not infected by influenza B.

The amount of recombinant HBc chimeric immunogen utilized in each immunization is referred to as an immunogenic effective amount and can vary widely, depending upon, e.g., the recombinant HBc chimeric immunogen, animal host immunized, and the presence of an adjuvant in the vaccine, as discussed below. Immunogenic effective amounts for a vaccine and an inoculum provide the protection or antibody activity, respectively, discussed hereinbefore.

Pharmaceutical compositions of the invention, such as vaccines or inocula, typically contain a recombinant HBc chimeric immunogen concentration of about 1 microgram to about 1 milligram per inoculation (unit dose), such as about 10 micrograms to about 50 micrograms per unit dose. (Immunizations in mice typically contain 10 or 20 μg of chimera particles.)

The term “unit dose” as it pertains to a pharmaceutical compositions of the present invention refers to a physically discrete unit suitable as a unitary dosage for animals, each unit containing a predetermined quantity of active material calculated to individually or collectively produce the desired immunogenic effect in association with the required diluent; i.e., carrier, or vehicle. A single unit dose or a plurality of unit doses can be used to provide an immunogenic effective amount of recombinant HBc chimeric immunogen particles.

Pharmaceutical compositions of the invention are typically prepared from recombinant HBc chimeric immunogen particles by dispersing the particles in a physiologically tolerable (acceptable) diluent vehicle such as water, saline phosphate-buffered saline (PBS), acetate-buffered saline (ABS), Ringer's solution or the like to form an aqueous composition. The diluent vehicle can also include oleaginous materials such as peanut oil, squalane, or squalene and alum, as is discussed hereinafter.

The immunogenically active ingredient is often mixed with excipients that are pharmaceutically acceptable and compatible with the active ingredient. Suitable excipients are, for example, water, saline, dextrose, glycerol, ethanol, or the like and combinations thereof. In addition, if desired, an inoculum or vaccine can contain minor amounts of auxiliary substances such as wetting or emulsifying agents, and pH buffering agents that enhance the immunogenic effectiveness of the composition. A pharmaceutical composition such as a vaccine or inoculum of the invention can optionally also include an adjuvant, as noted above. Suitable adjuvants for use in the present invention include those adjuvants that are capable of enhancing the antibody responses influenza sequences of the chimeras, as well as adjuvants capable of enhancing cell mediated responses towards T cell epitopes contained in the chimeras, if present. Adjuvants are well known in the art (see, for example, Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X).

Exemplary adjuvants include complete Freund's adjuvant (CFA), which is not used in humans, incomplete Freund's adjuvant (IFA), squalene, squalane and alum (e.g., Alhydrogel™ (Superfos, Denmark), which are materials well known in the art, and are available commercially from several sources.

Adjuvants for use with immunogens of the present invention include aluminum or calcium salts (for example hydroxide or phosphate salts). A specific example of an adjuvant for use herein is an aluminum hydroxide gel such as Alhydrogel™. For aluminum hydroxide gels (alum), the chimeric protein can be admixed with the adjuvant so that about 50 to about 800 micrograms of aluminum are present per dose, for example about 400 to about 600 micrograms are present. Calcium phosphate nanoparticles (CAP) is an adjuvant being developed by Biosante, Inc. (Lincolnshire, Ill.). The immunogen of interest can be either coated to the outside of particles, or encapsulated inside (He et al., Clin. Diagn. Lab. Immunol., 7(6):899-903, 2000).

Another adjuvant for use with an immunogen of the present invention is an emulsion. An exemplary emulsion is an oil-in-water emulsion or a water-in-oil emulsion. In addition to the immunogenic chimeric protein particles, such emulsions include an oil phase of squalene, squalane, peanut oil or the like as are well known, and a dispersing agent. Non-ionic dispersing agents can be used and such materials include mono- and di-C₁₂-C₂₄-fatty acid esters of sorbitan and mannide such as sorbitan mono-stearate, sorbitan mono-oleate, and mannide mono-oleate. An immunogen-containing emulsion is administered as an emulsion. Thus, in one example, such emulsions are water-in-oil emulsions that comprise squalene, glycerol, and a surfactant such as mannide mono-oleate (Arlacel™ A), optionally with squalane, emulsified with the chimeric protein particles in an aqueous phase. The oil phase can include about 0.1 to about 10 percent of the vaccine, such as about 0.2 to about 1 percent. Alternative components of the oil-phase include alpha-tocopherol, mixed-chain di- and tri-glycerides, and sorbitan esters. Well-known examples of such emulsions include Montanide™ ISA-720, and Montanide™ ISA 703 (Seppic, Castres, France). In one example, Montanide™ ISA-720 is used, and a ratio of oil-to-water of 7:3 (w/w) is used. Other oil-in-water emulsion adjuvants that can be used in the invention include those disclosed in WO 95/17210 and EP 0 399 843. The use of small molecule adjuvants is also contemplated herein. One type of small molecule adjuvant useful herein is a 7-substituted-8-oxo- or 8-sulfo-guanosine derivative described in U.S. Pat. No. 4,539,205, U.S. Pat. No. 4,643,992, U.S. Pat. No. 5,011,828, and U.S. Pat. No. 5,093,318, the disclosures of which are incorporated herein by reference. Of these materials, 7-allyl-8-oxoguanosine(loxoribine) has been shown to be particularly effective in inducing an antigen-(immunogen-) specific response.

Another useful adjuvant includes monophosphoryl lipid A (MPL®), 3-deacyl monophosphoryl lipid A (3D-MPL®), a well-known adjuvant manufactured by Corixa Corp. of Seattle, Wash., formerly Ribi Immunochem, Hamilton, Mont. The adjuvant contains three components extracted from bacteria: monophosphoryl lipid (MPL) A, trehalose dimycolate (TDM), and cell wall skeleton (CWS) (MPL+TDM+CWS) in a 2% squalene/Tween® 80 emulsion. This adjuvant can be prepared by the methods taught in GB 2122204B. An exemplary form of 3-de-O-acylated monophosphoryl lipid A is in the form of an emulsion having a small particle size less than 0.2 μm in diameter (EP 0 689 454 B1).

A further example is a compound structurally related to MPL® adjuvant called aminoalkyl glucosamide phosphates (AGPs) such as those available from Corixa Corp. under the designation RC-529® adjuvant {2-[(R)-3-tetra-decanoyloxytetradecanoylamino]-ethyl-2-deoxy-4-O-phosphon-o-3-O-[(R)-3-tetradecanoyloxytetra-decanoyl]-2-[(R)-3-tetra-decanoyloxytet-radecanoyl-amino]-p-D-glucopyranoside triethylammonium salt}.

An RC-529 adjuvant is available in a squalene emulsion sold as RC-529SE and in an aqueous formulation as RC-529AF available from Corixa Corp. (see U.S. Pat. No. 6,355,257 and U.S. Pat. No. 6,303,347; U.S. Pat. No. 6,113,918; and U.S. Publication No. 2003-0092643).

Further adjuvants that can be used in the invention include synthetic oligonucleotide adjuvants containing the CpG nucleotide motif one or more times (plus flanking sequences) available from Coley Pharmaceutical Group. The adjuvant designated QS21, available from Aquila Biopharmaceuticals, Inc., is an immunologically active saponin fraction having adjuvant activity derived from the bark of the South American tree Quillaja Saponaria Molina (e.g., Quil™ A), and the method of its production is disclosed in U.S. Pat. No. 5,057,540. Derivatives of Quil™ A, for example QS21 (an HPLC purified fraction derivative of Quil™ A also known as QA21), and other fractions such as QA17 are also disclosed. Semi-syntheic and synthetic derivatives of Quillaja Saponaria Molina saponins are also useful, such as those described in U.S. Pat. No. 5,977,081 and U.S. Pat. No. 6,080,725. The adjuvant denominated MF59 available from Chiron Corp. is described in U.S. Pat. No. 5,709,879 and U.S. Pat. No. 6,086,901.

Muramyl dipeptide adjuvants can also be used in the invention and include N-acetyl-muramyl-L-threonyl-D-isoglutamine (thur-MDP), N-acetyl-nor-muramyl-L-alanyl-D-isoglutamine [CGP 11637, referred to as nor-MDP], and N-acetylmuramyl-L-alanyl-D-isoglutaminyl-L-alanine-2-(1′-2′-dipalmityol-s-n-glycero-3-hydroxyphosphoryloxy)ethylamine [(CGP) 1983A, referred to as MTP-PE]. The so-called muramyl dipeptide analogues are described in U.S. Pat. No. 4,767,842.

Adjuvant mixtures that can be used in the invention include combinations of 3D-MPL and QS21 (EP 0 671 948 B1), oil-in-water emulsions comprising 3D-MPL and QS21 (WO 95/17210, PCT/EP98/05714), 3D-MPL formulated with other carriers (EP 0 689 454 B1), QS21 formulated in cholesterol-containing liposomes (WO 96/33739), and immunostimulatory oligonucleotides (WO 96/02555). Adjuvant SBAS2 (now ASO2), available from SKB (now Glaxo-SmithKline) contains QS21 and MPL in an oil-in-water emulsion, is also useful. Alternative adjuvants include those described in WO 99/52549 and non-particulate suspensions of polyoxyethylene ether (UK Patent Application No. 9807805.8).

An adjuvant that contains one or more agonists for toll-like receptor-4 (TLR-4) such as an MPL® adjuvant or a structurally related compound such as an RC-529® adjuvant or a Lipid A mimetic, alone or along with an agonist for TLR-9 such as a non-methylated oligo deoxynucleotide-containing the CpG motif can be used. Such adjuvants enhance the production of gamma-producing CD 8+, CD 4+ T cells and cytotoxic lymphocytes when admixed with a contemplated immunogenic HBc-containing particles or chemically linked to such an immunogen. Alum also can be present in such an adjuvant mixture.

A further adjuvant mixture that can be used in the invention includes a stable water-in-oil emulsion further containing aminoalkyl glucosamine phosphates such as described in U.S. Pat. No. 6,113,918. Of the aminoalkyl glucosamine phosphates, the molecule known as RC-529 {(2-[(R)-3-tetradecanoyloxytetradecanoylamino]ethyl 2-deoxy-4-O-phosphono-3-O-[(R)-3-tetradecanoyloxy-tetradecanoyl]-2-[(R)-3-tetradecanoyloxytetra-decanoylamino]-p-D-glucopyranoside triethylammonium salt)} is one example. An exemplary water-in-oil emulsion is described in WO 99/56776.

Adjuvants are utilized in an adjuvant amount, which can vary with the adjuvant, host animal and recombinant HBc chimeric immunogen. Typical amounts can vary from about 1 μg to about 1 mg per immunization. Those skilled in the art know that appropriate concentrations or amounts can be readily determined.

Pharmaceutical compositions such as inocula and vaccines are conventionally administered parenterally, by injection, for example, either subcutaneously, intradermally, or intramuscularly. Additional formulations that are suitable for other modes of administration include suppositories and, in some cases, oral formulation or by nasal spray. For suppositories, traditional binders and carriers can include, for example, polyalkalene glycols or triglycerides; such suppositories may be formed from mixtures containing the active ingredient in the range of 0.5% to 10%, e.g., 1-2%. Oral formulations include such normally employed excipients as, for example, pharmaceutical grades of mannitol, lactose, starch, magnesium stearate, sodium saccharine, cellulose, magnesium carbonate and the like.

A pharmaceutical composition such as an inoculum or vaccine composition can take the form of a solution, suspension, tablet, pill, capsule, sustained release formulation or powder, and contains an immunogenic effective amount of HBc chimera, such as in the form of particles, as active ingredient. In a typical composition, an immunogenic effective amount of HBc chimeric particles is about 1 μg to about 1 mg of active ingredient per dose, such as about 5 μg to about 50 μg per dose, as noted above. A pharmaceutical composition such as a vaccine or inoculum is typically formulated for intranasal (IN) or parenteral administration. Exemplary immunizations are carried out sub-cutaneously (SC) intra-muscularly (IM), intravenously (IV), intraperitoneally (IP) or intra-dermally (ID).

The HBc chimera particles and HBc chimera particle conjugates can be formulated as neutral or salt forms. Pharmaceutically acceptable salts include the acid addition salts (formed with the free amino groups of the protein or hapten) and are formed with inorganic acids such as, for example, hydrochloric or phosphoric acids, or such organic acids as acetic, oxalic, tartaric, mandelic, and the like. Salts formed with the free carboxyl groups can also be derived form inorganic bases such as, for example, sodium, potassium, ammonium, calcium, or ferric hydroxides, and such organic bases as isopropylamine, trimethylamine, 2-ethylamino ethanol, histidine, procaine, and the like.

The pharmaceutical compositions are administered in a manner compatible with the dosage formulation, and in such amount as is therapeutically effective and immunogenic (an antibody-inducing amount or protective amount, as is desired). The quantity to be administered depends on the subject to be treated, capacity of the subject's immune system to synthesize antibodies, and degree of protection desired. Precise amounts of active ingredient required to be administered depend on the judgment of the practitioner and can be peculiar to each individual. However, suitable dosage ranges are of the order of several hundred micrograms active ingredient per individual. Suitable regimes for initial administration and booster shots are also variable, but are typified by an initial administration followed in intervals (weeks or months) by a subsequent injection or other administration.

Once immunized, the host animal is maintained for a period of time sufficient for the recombinant HBc chimeric immunogen to induce the production of a sufficient titer of antibodies that bind to the M2 protein. The maintenance time for the production of anti-M2 antibodies typically lasts for a period of about three to about twelve weeks, and can include a booster, second immunizing administration of the vaccine. A third immunization is also contemplated, if desired, at a time several weeks to five years after the first immunization. It is particularly contemplated that once a protective level titer of antibodies is attained, the vaccinated host animal is preferably maintained at or near that antibody titer by periodic booster immunizations administered at intervals of about 1 to about 5 years.

The production of antibodies can be readily ascertained by obtaining a plasma or serum sample from the immunized host and assaying the antibodies therein for their ability to bind to a synthetic polypeptide antigen in an immunoassay such as an ELISA assay a Western blot as is well known in the art.

It is noted that the above-described antibodies so induced can be isolated from the blood of the host using well-known techniques, and then reconstituted into a second vaccine for passive immunization as is also well known. Similar techniques are used for gamma-globulin immunizations of humans. For example, antiserum from one or a number of immunized hosts can be precipitated in aqueous ammonium sulfate (typically at 40-50 percent of saturation), and the precipitated antibodies purified chromatographically as by use of affinity chromatography in which a relevant influenza B polypeptide is utilized as the antigen immobilized on the chromatographic column.

Without further elaboration, it is believed that one skilled in the art can, using the preceding description and the detailed examples below, utilize the present invention to its fullest extent. The following specific embodiments are, therefore, to be construed as merely illustrative, and not limiting of the remainder of the disclosure in any way whatsoever.

### Experimental Examples

HA0-based Influenza B vaccine

HA is composed of two subunits, HA1 (molecular mass of 55 kDa) and HA2 (molecular mass of 25 kDa), which are cleaved by host proteases from a precursor, HA0 (molecular mass of 75 kDa) (Skehel et al., Proc. Natl. Acad. Sci. U.S.A. 72:93-97, 1975). The cleavage of HA0 into HA1/HA2 activates virus infectivity (Klenk et al., Virology 68:426-439, 1975) and is important for influenza virus pathogenicity in human and avian hosts (Klenk et al., Trends Microbiol. 2:39-43, 1994). The major characteristic of HA that determines sensitivity to host proteases is the composition of the proteolytic cleavage site in the external loop of the HA0 molecule, which links HA1 and HA2 (Chen et al., Cell 95:409-417, 1998). This loop may contain either a single Arg or Lys residue (monobasic cleavage site) or several Lys and/or Arg residues, with an R-X-K/R-R motif, which forms a multibasic cleavage site.

We generated multiple versions of HBc constructs harboring an HA0 peptide (PAKLLKERGFFGAIAGFLE) in major immunogenic sites of HBc and characterized them in vitro for solubility and formation of VLPs. Several chemically synthesized BHA0 peptides conjugated to HBc and/or KLH were also made. For the latter purpose, a PAKLLKERGFFGAIAGFLEGSGC synthetic peptide was used (see FIG. 2).

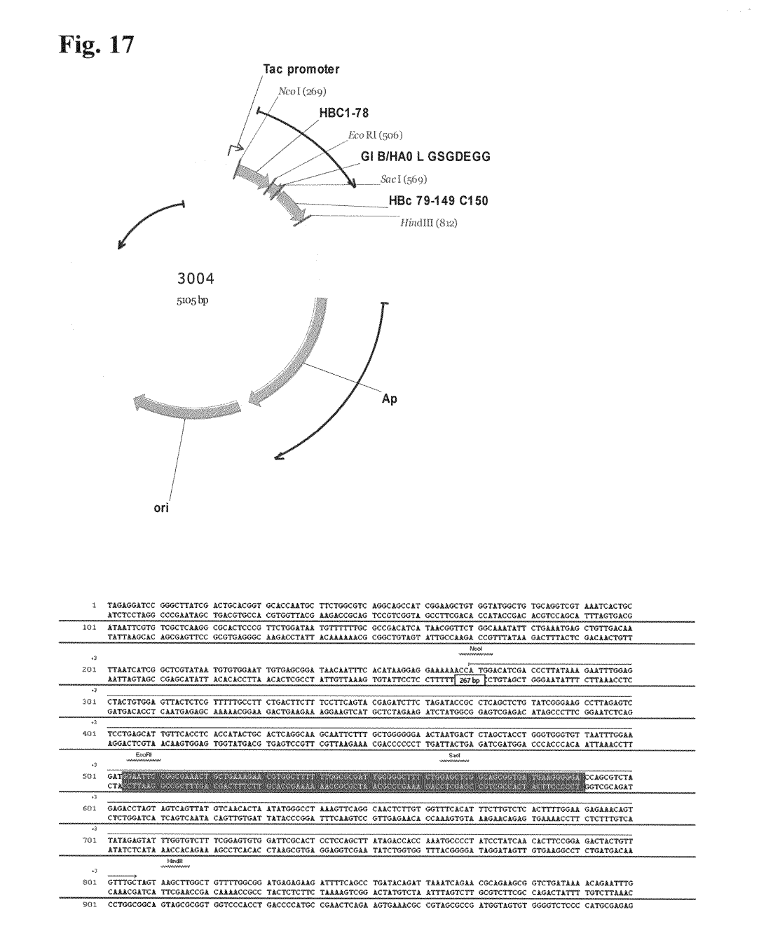

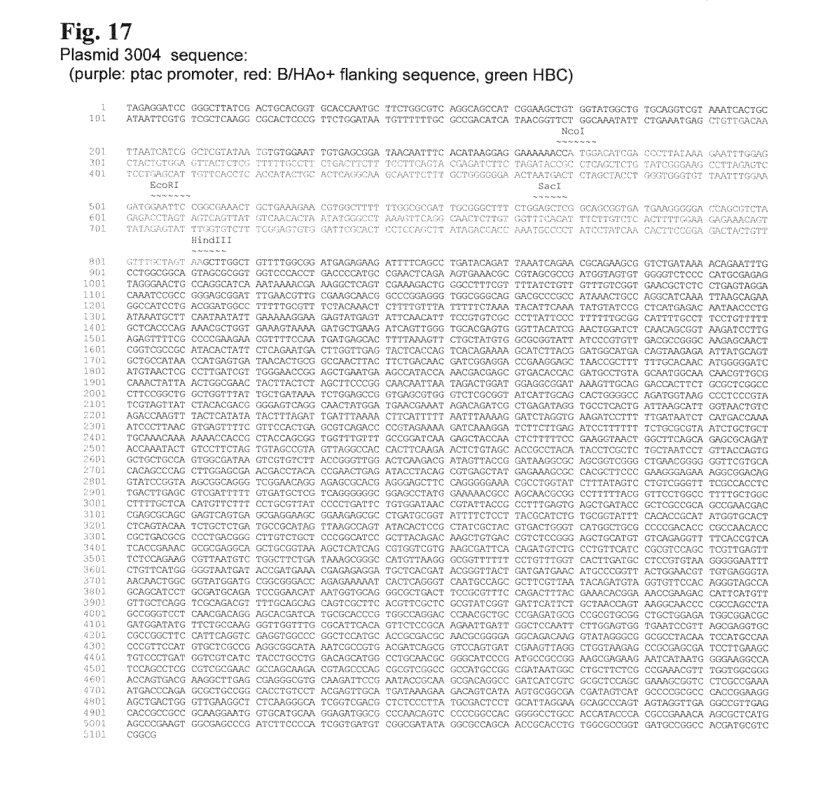

Six out of thirty fusion constructs demonstrated expression of soluble VLPs. To increase solubility of the target particles, we applied a technique named “hybrid VLPs,” exploiting simultaneous expression of “wild-type” HBc (without insert) and HBc-HA0 in the same bacterial cell. HBc and HBc-BHA0 monomers were either expressed from a single plasmid (FIG. 2) or from two plasmids driven from different promoters. Soluble VLPs 3004, 3010, and 3009 (Table 1; see FIG. 17 for additional details of 3004 construct) were purified and tested for immunogenicity and protection in a murine non-lethal model of influenza B infection. As a control, an HA0 peptide chemically conjugated to HBc (1843-BHA0) was prepared. Generation of 1843 particles and the chemical cross-linking process were done as described in U.S. Pat. No. 6,231,864 B1.

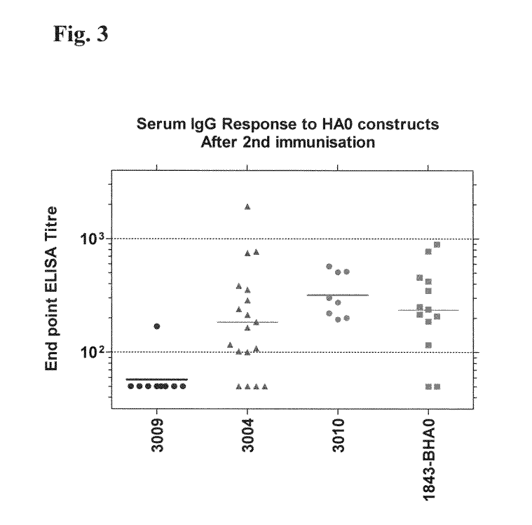

As is shown in FIG. 3, the BHA0 peptide induces anti-HA0 antibody responses when presented as a genetically inserted peptide into the major immunogenic region (MIR) of HBc or as a chemically conjugated linear peptide (1843-BHA0).

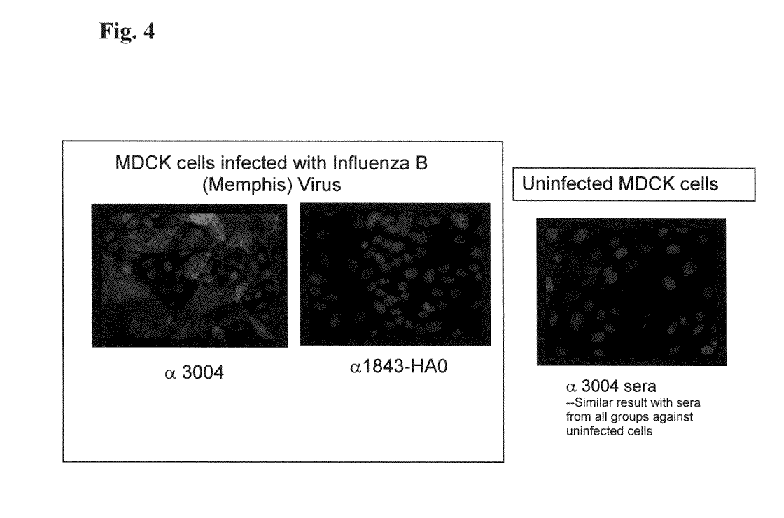

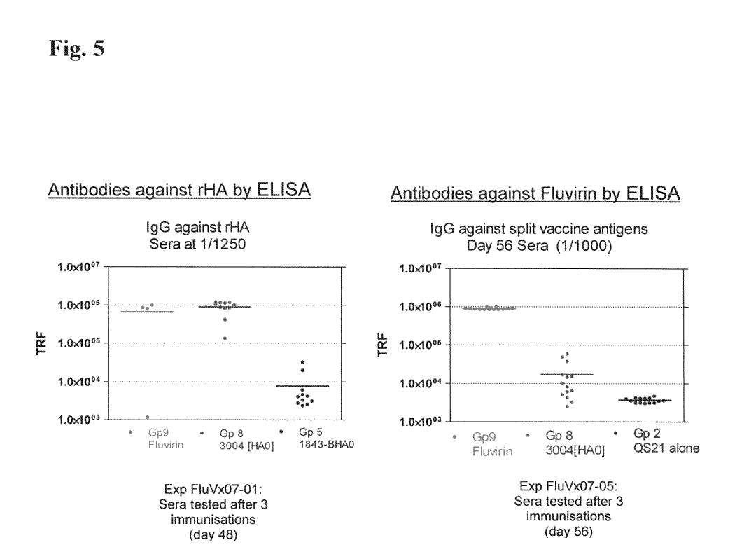

As is shown in FIG. 4, the BHA0 loop insertion (3004) is superior to the chemically-linked peptide at inducing antibodies reactive with infected cells. MDCK cells were seeded on chamber slides and either infected with influenza B (Memphis strain) or left uninfected as a control. Serum from mice immunized with various constructs were pooled and used at 1:200 to visualize virus-infected cells. Mouse IgG was detected using goat anti-mouse Alexa 488 and visualized using the Axioskop2 by Zeiss.

As shown in FIG. 5, the BHA0 loop insertion induces antibodies that are cross-reactive with recombinant and native HA. Antibodies generated against 3004 and 1843-BHA0 were found to be immunoreactive with recombinant or native HA.

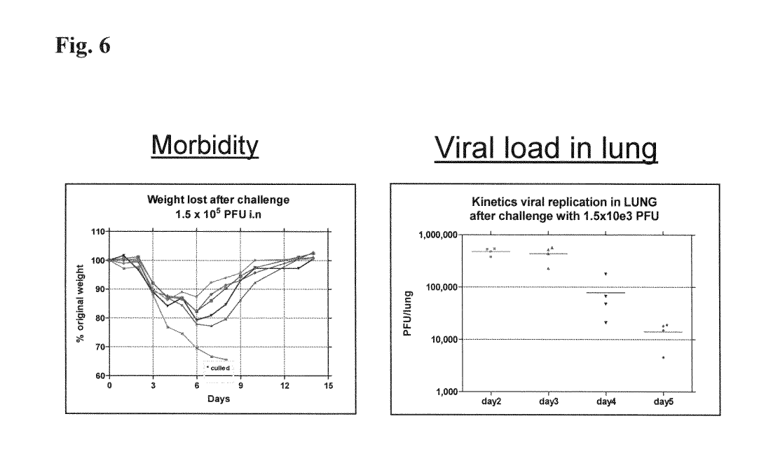

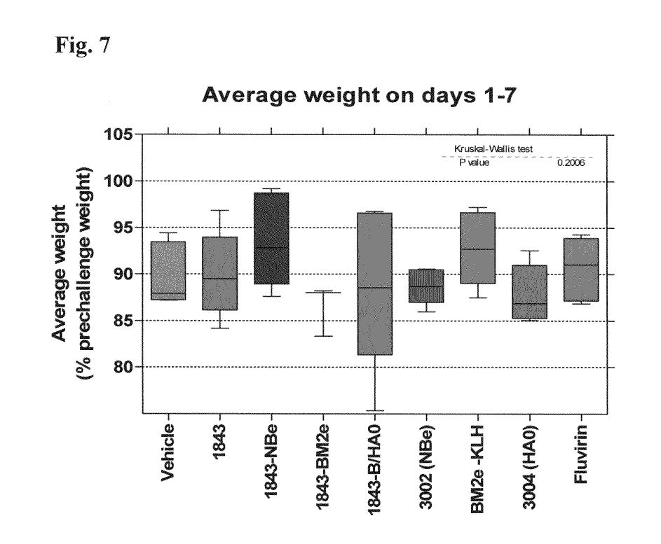

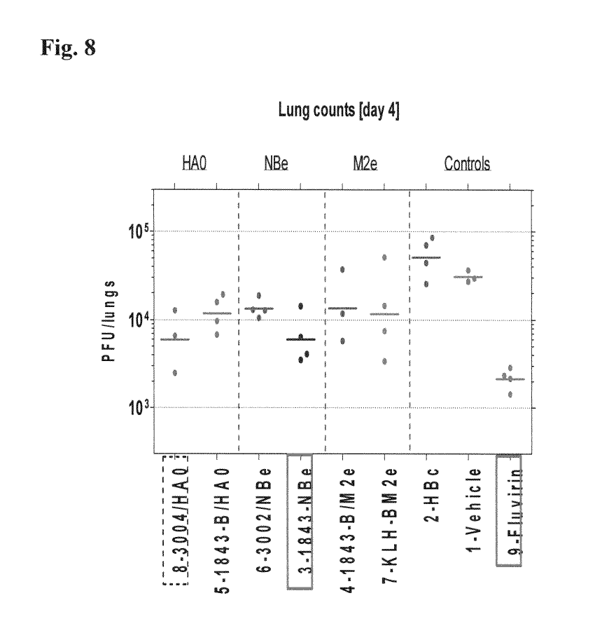

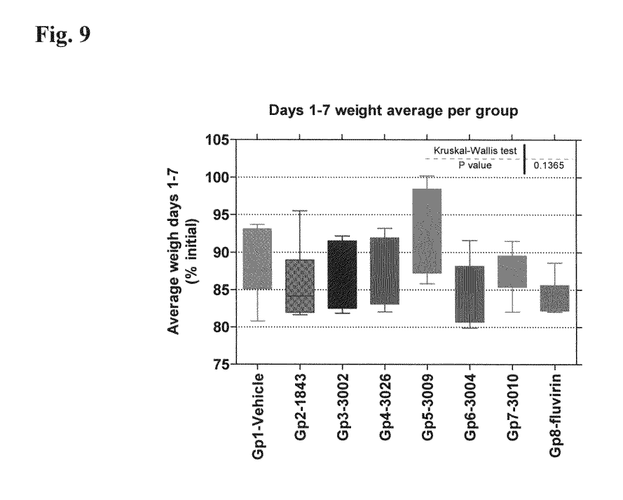

To establish a murine challenge model, Memphis/12/97 (M15) virus was serially passaged in mice 6 times. As is shown in FIG. 6, the resulting virus resulted in significant morbidity, but all mice survived, which was correlated with reduced virus loads in lungs. Briefly, mice were immunized on three occasions (days 0, 21, and 42) with the particles and QS21 adjuvant, and challenged by the intranasal route with 1.5×10⁵ of adapted influenza B/Memphis/12/97 (M15) strain on day 63. No significant difference between any particles containing BHA0 and mock-immunized mice with regard to weight lost after challenge (FIG. 7) was detected. However, lung counts (FIG. 8) in groups immunized with Fluvirin® (subunit influenza vaccine) showed a significant reduction in viral load in lungs, as compared to the mock-immunized control. The group immunized with 3004 also showed a reduction in viral load, as compared to the control, but the difference was not statistically significant.

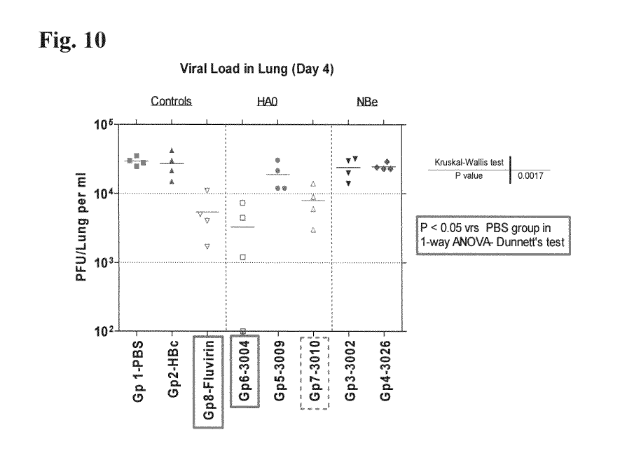

A second in vivo experiment was performed in a reduced fashion (two immunizations instead of three and 10³ pfu of challenge virus per mouse). As is shown in FIG. 9, there was no significant difference between HBAO-immunized mice and mock-immunized mice with regard to weight lost after challenge. However, lung counts (FIG. 10) in groups immunized with 3004 showed a significant reduction in viral load in lungs, as compared to mock-immunized controls. Reduction was comparable to a Fluvirin®(subunit influenza vaccine)-immunized group of mice. The group immunized with 3010 also showed a reduction in viral load, as compared to the control, but the difference was not statistically significant.

In summary, these data show that HBc-BHA0 VLPs are highly immunogenic in either the form of a chemical conjugate or a genetic fusion. Both forms were able to induce antibodies in mice that cross-react with native and recombinant HA in vitro (ELISA and MDCK experiments). Two challenge experiments demonstrated a reduction of viral load in lungs of mice immunized with a genetic fusion HBc-BHA0. The reduction was shown to be statistically significant when the challenge dose was 10³ pfu per mouse. Thus, HBc-BHA0 biological fusions were shown to be efficient vaccine candidates against influenza B infection.

### NBe-based Influenza B Vaccine

The NB glycoprotein of influenza B virus belongs to a class of integral membrane proteins of 100 amino acids, has an apparent molecular weight of 18,000 on polyacrylamide gels, is abundantly synthesized in influenza B virus-infected cells (Shaw et al., Proc. Natl. Acad. Sci. U.S.A. 80:4879-4883, 1983), and its function is related to cation-selective channels (Sunstrom et al., J. Membr. Biol. 150:127-132, 1996). From both biochemical and genetic data, it has been shown that NB has an N-terminal extracellularly exposed domain and a relatively large C-terminal domain that is intracellular. The approximately 18 amino acid N-terminal region of NB (NBe) has been shown to be highly conserved within influenza B strains and contains two N-linked carbohydrate chains of the high-mannose form attached to asparagines at residues 3 and 7 (Betakova et al., J. Gen. Virol. 77 (Pt 11):2689-2694, 1996). The NB protein is a product of RNA segment 6 of influenza B viruses and its open reading frame overlaps with that of neuraminidase (NA). The high conservation of the extracellular domain implies that its structure is essential for biological function of influenza B virus.

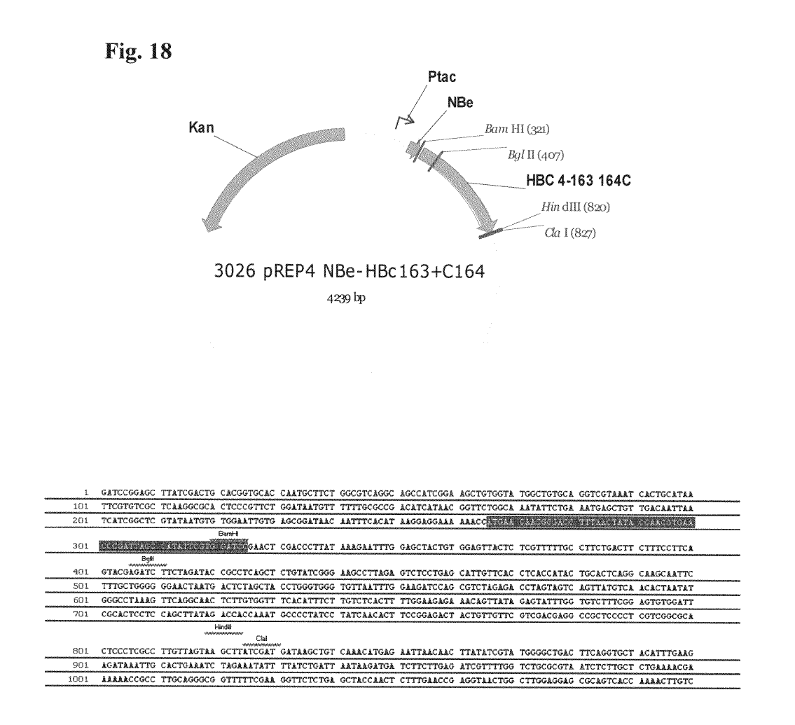

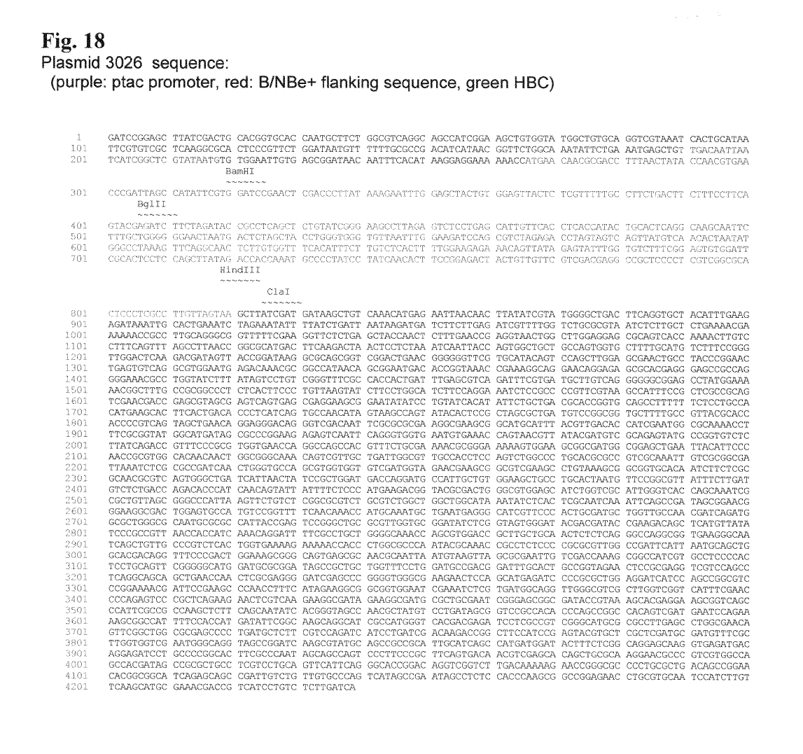

To mimic the natural location of the NBe peptide (NNATFNYTNVNPISHIRGS) in NB, we fused it N-terminally to HBc150 and HBc163, which resulted in two constructs, 3002 (HBc150-NBe) and 3026 (HBc163-NBe) (FIG. 11) (see FIGS. 16 and 18 for further details of constructs 3002 and 3026).

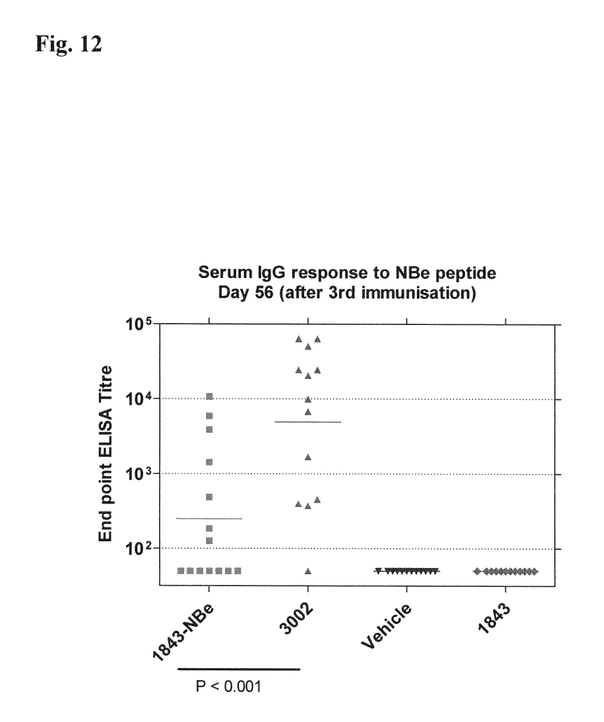

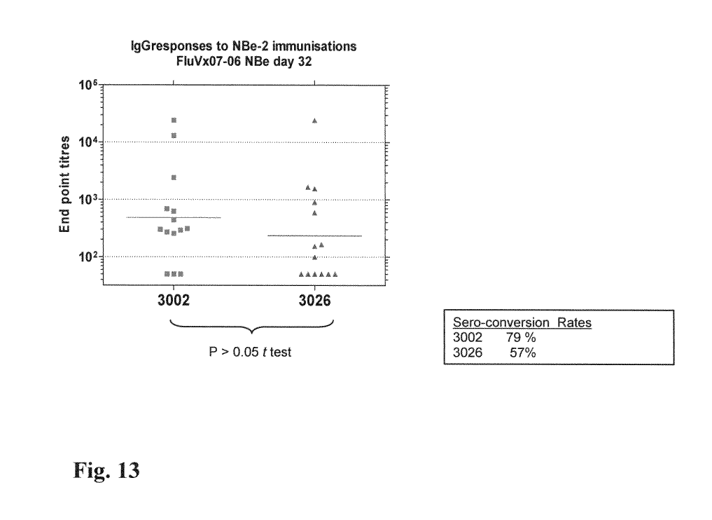

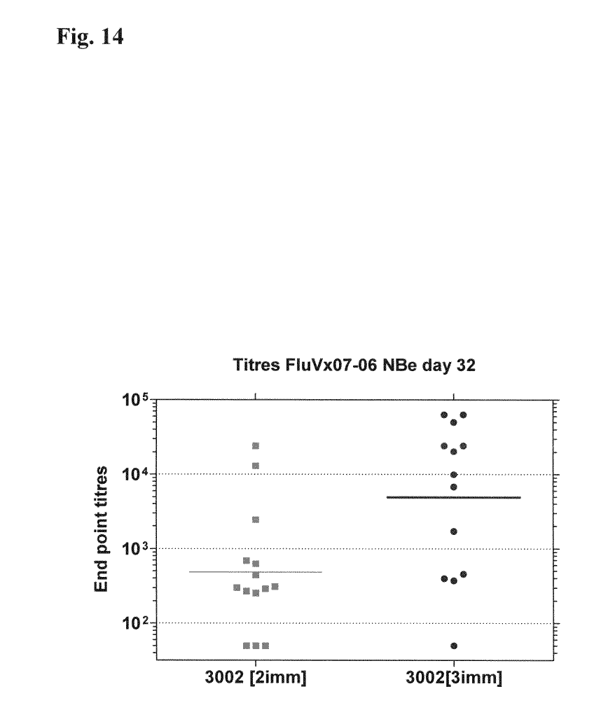

Genetic fusion 3002 was shown to be superior to a chemical conjugate of NBe for immunogenicity (FIG. 12). Constructs 3002 and 3026 induced similar antibody titers, but responses against the 3002 construct were more consistent (FIG. 13), and responses to the 3002 construct was shown to be dose-dependent (FIG. 14).

In a first challenge experiment (see description of HA0-based influenza B vaccine, above) mice, immunized with 1843-NBe or with Fluvirin showed significant reductions in viral load in the lungs, as compared to mock-immunized controls (FIG. 8).

In summary, HBc-NBe was shown to be highly immunogenic in mice. The chemical conjugate was shown to be efficacious: after three immunizations with 1843-NBe viral load in lungs of infected mice was significantly reduced.

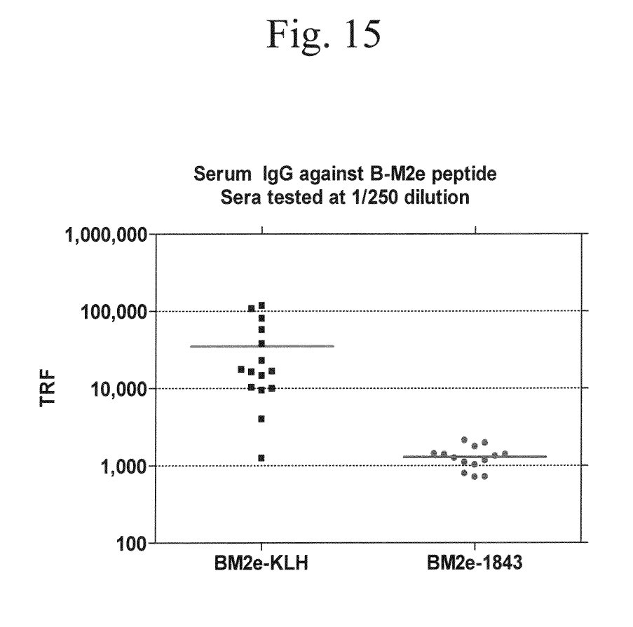

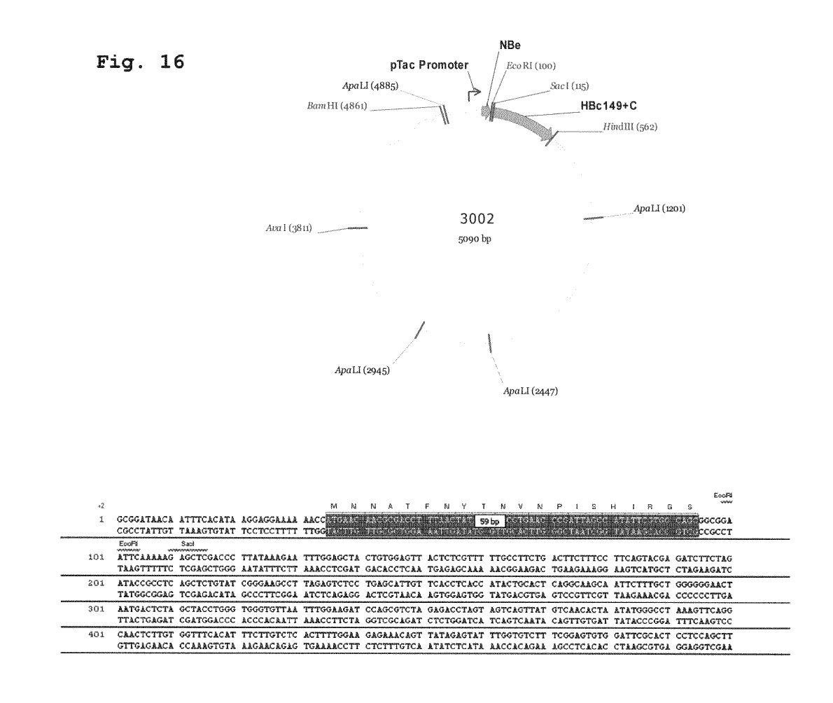

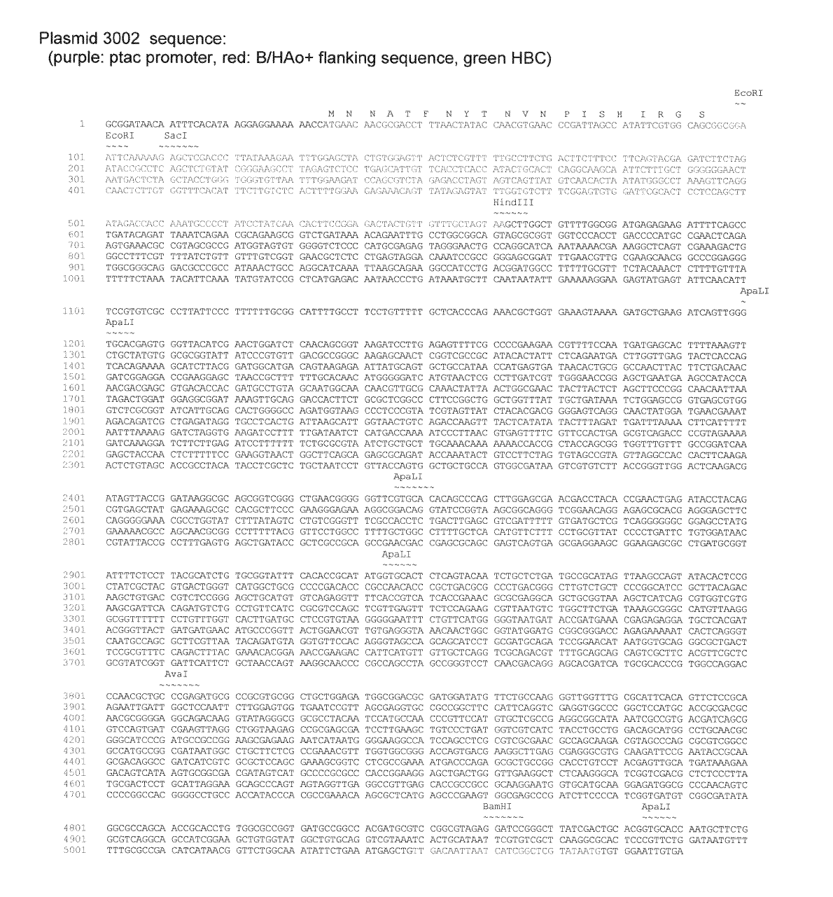

FIG. 15 shows the results of immunogenicity tests of other compositions of the invention, BM2e-KLH and BM2e-1843 (an HBc-based chemical conjugate). Mice were immunized three times (days 0, 21, and 42) with the constructs and QS21. Blood samples were tested on day 56.

TABLE 1
HBc-HBA0 constructs used for in vivo studies

| Name of the | | |
|---|---|---|
| construct | Proteins | Construct characteristics |
| 3004 | HBc150-HA0 + | Proteins expressed in E. coli from |
| | HBc150 | two coexisting plasmids |
| 3010 | HBc150-HA0 + | Proteins expressed in BLR E. coli |
| | HBc150 | from one plasmid as separate ORFs |
| | | from different tac promoters |
| 3009 | HBc150-HA0 + | Proteins expressed from in BLR E. coli |
| | HBc163 | strain from two coexisting |
| | | plasmids |
| 1843-BHAo | HBc150-HA0 | HAo peptide chemically linked to |
| | | HBC-150 VLPs |

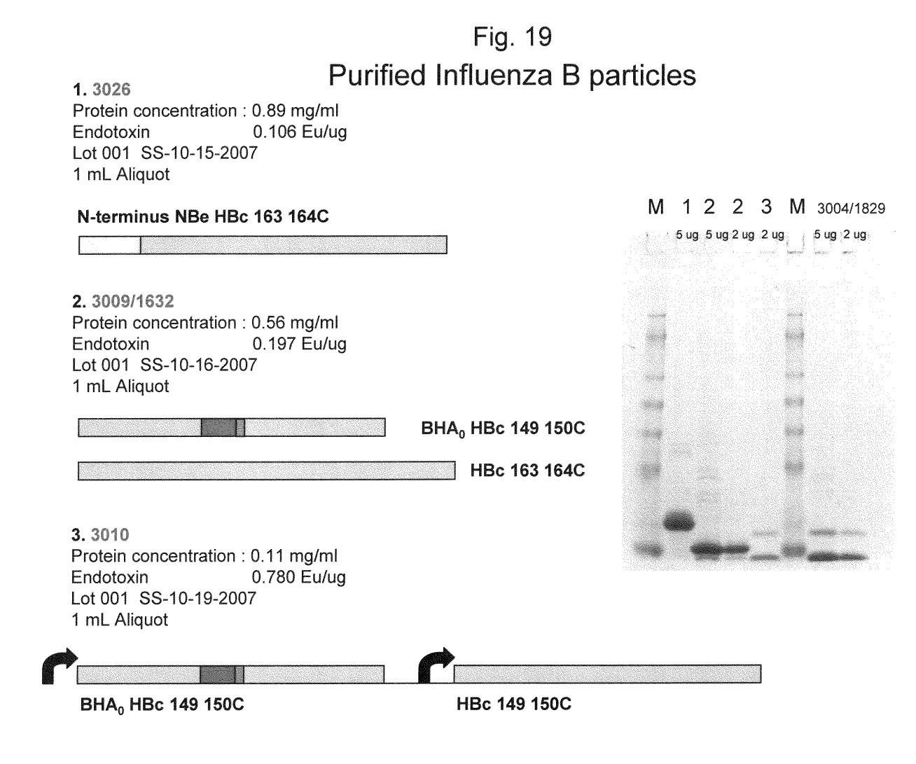

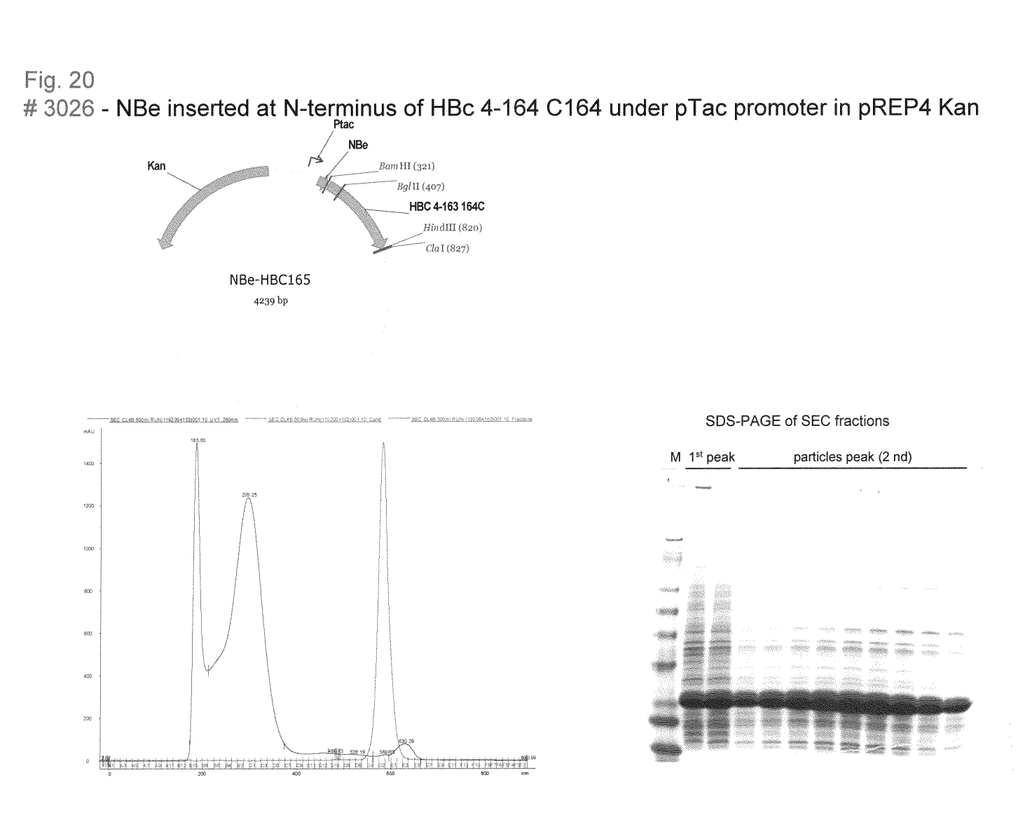

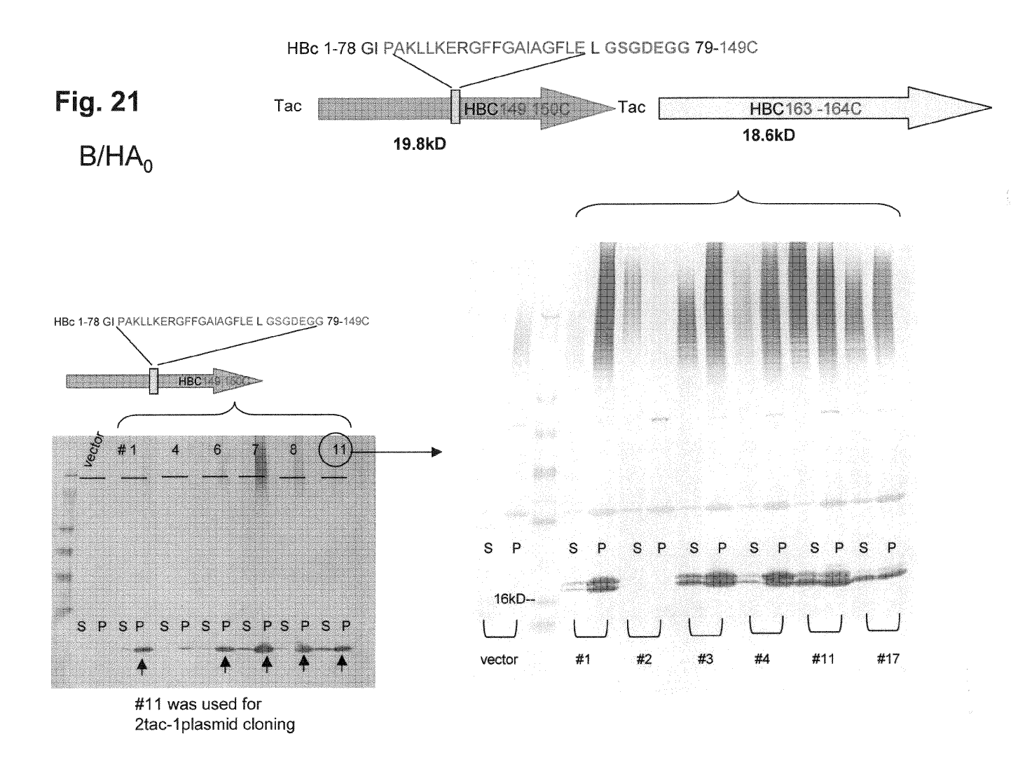

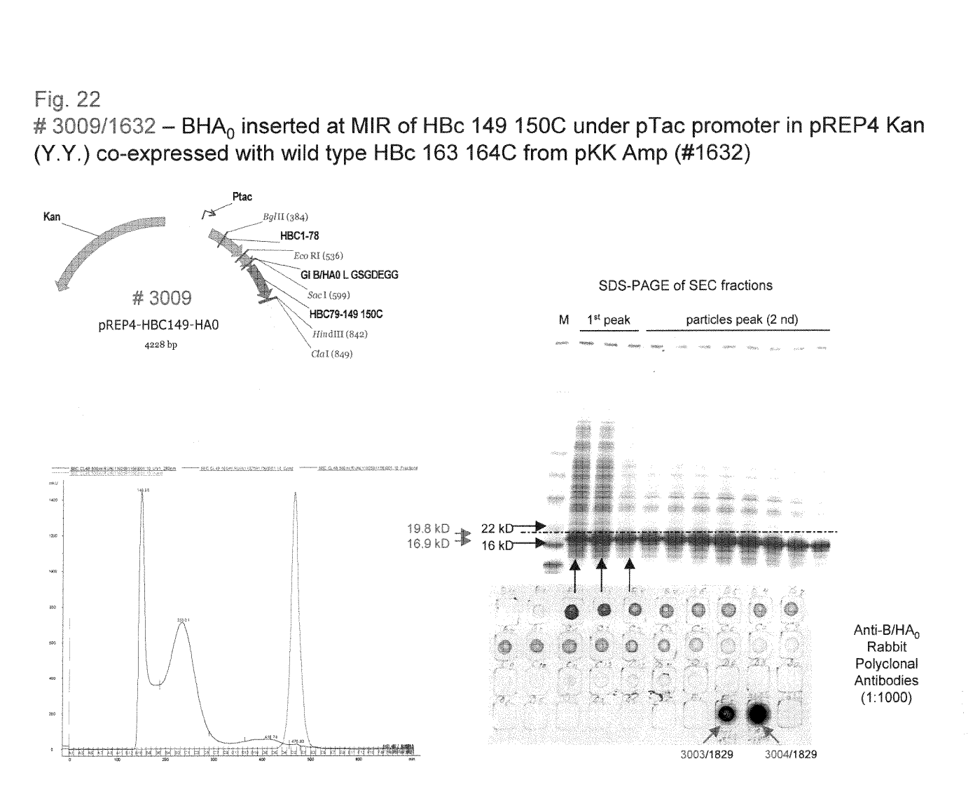

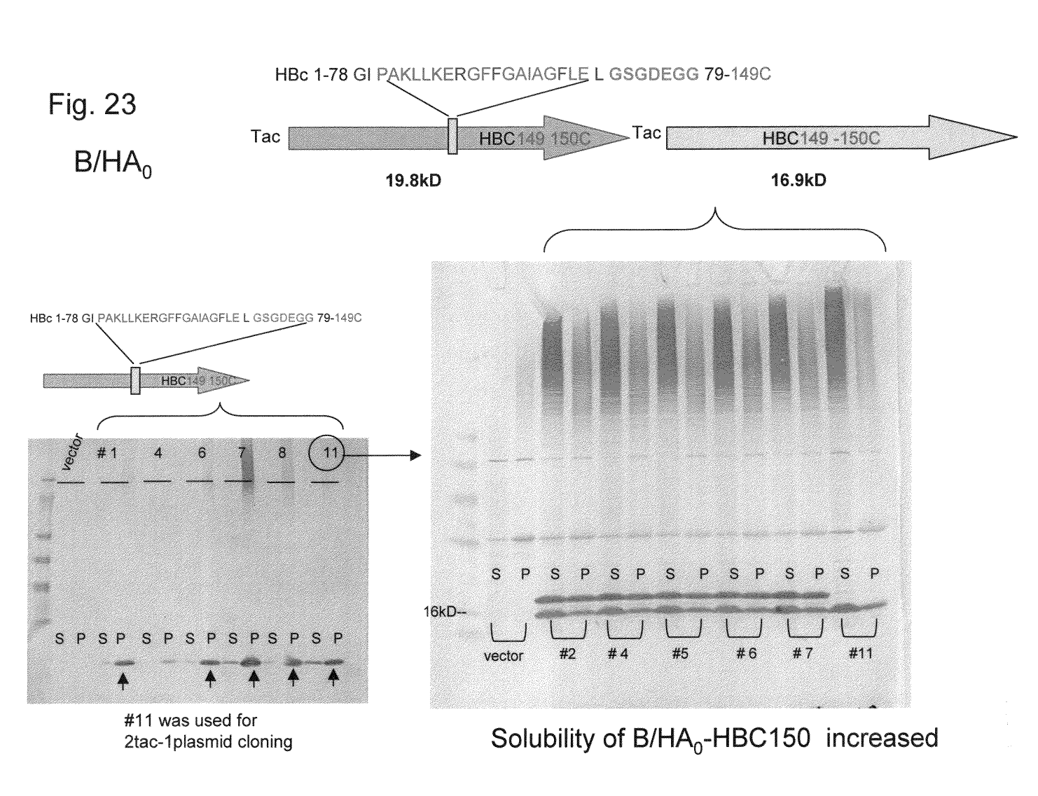

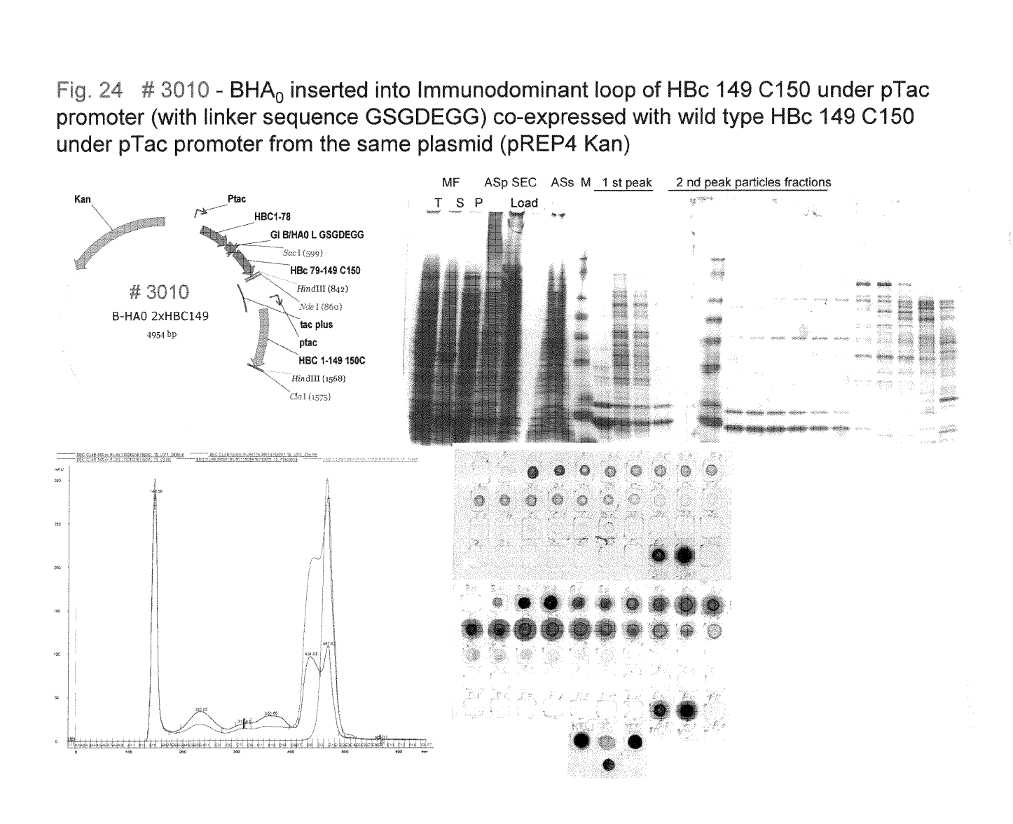

*HBc150 and HBc163 signify the length of HBC monomer 150 amino acids or 165 amino acids, respectively
** Full sequences of all plasmids are included in FIGS. 16-18. FIGS. 19-24 include additional data concerning purification of HBc-influenza B protein particles according to the invention.

The sequences of the HBc molecules shown in FIG. 1 are as follows:

HBcAYW DNA
SEQ ID NO: 7
atggacatcg acccttataa agaatttgga gctactgtgg 60
agttactctc gtttttgcct
tctgacttct ttccttcagt acgagatctt ctagataccg 120
cctcagctct gtatcgggaa
gccttagagt ctcctgagca ttgttcacct caccatactg 180
cactcaggca agcaattctt
tgctgggggg aactaatgac tctagctacc tgggtgggtg 240
ttaatttgga agatccagcg
tctagagacc tagtagtcag ttatgtcaac actaatatgg 300
gcctaaagtt caggcaactc
ttgtggtttc acatttcttg tctcactttt ggaagagaaa 360
cagttataga gtatttggtg
tctttcggag tgtggattcg cactcctcca gcttatagac 420
caccaaatgc ccctatccta
tcaacacttc cggagactac tgttgttaga cgacgaggca 480
ggtcccctag aagaagaact
ccctcgcctc gcagacgaag gtctcaatcg ccgcgtcgca 540
gaagatctca atctcgggaa
tctcaatgt
HBcADW DNA
SEQ ID NO: 8
atggacattg acccttataa agaatttgga gctactgtgg 60
agttactctc gtttttgcct
tctgacttct ttccttccgt acgagatctc ctagacaccg 120
cctcagctct gtatcgagaa
gccttagagt ctcctgagca ttgctcacct caccatactg 180
cactcaggca agccattctc
tgctgggggg aattgatgac tctagctacc tgggtgggta 240
ataatttgca agatccagca
tccagagatc tagtagtcaa ttatgttaat actaacatgg 300
gtttaaagat caggcaacta
ttgtggtttc atatatcttg ccttactttt ggaagagaga 360
ctgtacttga atatttggtc
tctttcggag tgtggattcg cactcctcca gcctatagac 420
caccaaatgc ccctatctta
tcaacacttc cggaaactac tgttgttaga cgacgggacc 480
gaggcaggtc ccctagaaga
agaactccct cgcctcgcag acgcagatct caatcgccgc 540
gtcgcagaag atctcaatct
cgggaatctc aatgt
HBcADW2 DNA
SEQ ID NO: 9
atggacattg acccttataa agaatttgga gctactgtgg 60
agttactctc gtttttgcct
tctgacttct ttccttccgt cagagatctc ctagacaccg 120
cctcagctct gtatcgagaa
gccttagagt ctcctgagca ttgctcacct caccatactg 180
cactcaggca agccattctc
gctgggggg aattgatgac tctagctacc tgggtgggta 240
ataatttgga agatccagca
tctagggatc ttgtagtaaa ttatgttaat actaacgtgg 300
gtttaaagat caggcaacta
ttgtggtttc atatatcttg ccttactttt ggaagagaga 360
ctgtacttga atatttggtc
tctttcggag tgtggattcg cactcctcca gcctatagac 420
caccaaatgc ccctatctta
tcaacacttc cggaaactac tgttgttaga cgacgggacc 480
gaggcaggtc ccctagaaga
agaactccct cgcctcgcag acgcagatct ccatcgccgc 540
gtcgcagaag atctcaatct
cgggaatctc aatgt
HBcADYW DNA
SEQ ID NO: 10
atggacattg acccttataa agaatttgga gctactgtgg 60
agttactctc gtttttgcct
tctgacttct ttccttccgt acgagatctt ctagataccg 120
ccgcagctct gtatcgggat
gccttagagt ctcctgagca ttgttcacct caccatactg 180
cactcaggca agcaattctt
tgctggggag acttaatgac tctagctacc tgggtgggta 240
ctaatttaga agatccagca
tctagggacc tagtagtcag ttatgtcaac actaatgtgg 300
gcctaaagtt cagacaatta
ttgtggtttc acatttcttg tctcactttt ggaagagaaa 360
cggttctaga gtatttggtg
tcttttggag tgtggattcg cactcctcca gcttatagac 420
caccaaatgc ccctatccta
tcaacgcttc cggagactac tgttgttaga cgacgaggca 480
ggtcccctag aagaagaact
ccctcgcctc gcagacgaag atctcaatcg ccgcgtcgca 540
gaagatctca atctcgggaa
tctcaatgt
Woodchuck DNA
SEQ ID NO: 11
atggctttgg ggcatggaca tagatcctta taaagaattt 60
ggttcatctt atcagttgtt
gaattttctt cctttggact tctttcctga tcttaatgct 120
ttggtggaca ctgctactgc
cttgtatgaa gaagaactaa caggtaggga acattgctct 180
ccgcaccata cagctattag
acaagcttta gtatgctggg atgaattaac taaattgata 240
gcttggatga gctctaacat
aacttctgaa caagtaagaa caatcattgt aaatcatgtc 300
aatgatacct ggggacttaa
ggtgagacaa agtttatggt ttcatttgtc atgtctcact 360
ttcggacaac atacagttca
agaattttta gtaagttttg gagtatggat caggactcca 420
gctccatata gacctcctaa
tgcacccatt ctctcgactc ttccggaaca tacagtcatt 480
aggagaagag gaggtgcaag
agcttctagg tcccccagaa gacgcactcc ctctcctcgc 540
aggagaagat ctcaatcacc
gcgtcgcag
Ground Squirrel DNA
SEQ ID NO: 12
atgtatcttt ttcacctgtg ccttgttttt gcctgtgttc 60
catgtcctac tgttcaagcc
tccaagctgt gccttggatg gctttgggac atggacatag 120
atccctataa agaatttggt
tcttcttatc agttgttgaa ttttcttcct ttggactttt 180
ttcctgatct caatgcattg
gtggacactg ctgctgctct ttatgaagaa gaattaacag 240
gtagggagca ttgttctcct
catcatactg ctattagaca ggccttagtg tgttgggaag 300
aattaactag attaattaca
tggatgagtg aaaatacaac agaagaagtt agaagaatta 360
ttgttgatca tgtcaataat
acttggggac ttaaagtaag acagacttta tggtttcatt 420
tatcatgtct tacttttgga
caacacacag ttcaagaatt tttggttagt tttggagtat 480
ggattagaac tccagctcct
tatagaccac ctaatgcacc cattttatca actcttccgg 540
aacatacagt cattaggaga
agaggaggtt caagagctgc taggtccccc cgaagacgca 600
ctccctctcc tcgcaggaga
aggtctcaat caccgcgtcg cagacgctct caatctccag 651
cttccaactg c

### Other Embodiments

All publications, patent applications, and patents mentioned in this specification are incorporated herein by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention. Use of singular forms herein, such as “a” and “the,” does not exclude indication of the corresponding plural form, unless the context indicates to the contrary.

What is claimed is:

1. A polypeptide comprising hepatitis B core protein sequences and influenza B virus sequences.

2. The polypeptide of claim 1, wherein said influenza B virus sequences comprise hemagglutinin precursor protein sequences.

3. The polypeptide of claim 1, wherein said influenza B virus sequences comprise NB sequences.

4. The polypeptide of claim 3, wherein said NB sequences comprise NBe sequences.

5. The polypeptide of claim 1, wherein said hepatitis B core protein sequences comprise a carboxy-terminal truncation.

6. The polypeptide of claim 5, wherein said carboxy-terminal truncation is after amino acid 149, 150, 163, or 164 of hepatitis B core protein.

7. The polypeptide of claim 1, wherein said influenza B virus sequences are inserted in the major immunodominant region (MIR) of said hepatitis B core protein sequences.

8. The polypeptide of claim 7, wherein said influenza B virus sequences are inserted in the region of amino acids 75-83 of said hepatitis B core protein sequences.

9. The polypeptide of claim 1, wherein said influenza B virus sequences are inserted at the amino terminus of said hepatitis B core protein.

10. The polypeptide of claim 1, wherein said hepatitis B core and influenza B virus sequences are chemically-linked.

11. The polypeptide of claim 1, wherein the recombinant hepatitis B virus core (HBc) protein comprises Domains I, II, III, and IV as described herein.

12. A virus-like particle comprising a polypeptide of claim 1.

13. The virus-like particle of claim 12, further comprising hepatitis B core sequences lacking the insertion or chemical linkage of influenza B virus sequences.

14. A nucleic acid molecule encoding the polypeptide of claim 1.

15. A pharmaceutical composition comprising the polypeptide of claim 1.

16. The pharmaceutical composition of claim 15, further comprising an adjuvant.

17. A method of inducing an immune response to influenza virus B in a subject, the method comprising administering to the subject the polypeptide of claim 1.

18. The method of claim 17, wherein said subject does not have, but is at risk of acquiring, an influenza B virus infection.

19. The method of claim 17, wherein said subject has influenza B virus infection.

20. The method of claim 17 further comprising administering to the subject a second, different immunological agent against an influenza virus.
